SEQUENCE LISTING (1 sequence)

SEQ ID NO: 1 - protein, 20 aa, Artificial
  Synthetic Peptide
  Xaa (1)..(1): Xaa is B-Ala
  Xaa (2)..(2): Xaa is Sar10
  Xaa (4)..(4): Xaa is HArg
  Xaa (7)..(7): Xaa is HyP
  Xaa (19)..(19): Xaa is HArg
XXAXDCXLVNPLCLHPDWXC

(19) United States
(12) Patent Application Publication
(10) Pub. No.: US 2023/0025916 A1
(43) Pub. Date: Jan. 26, 2023

(54) BICYCLIC PEPTIDE LIGANDS SPECIFIC FOR EphA2 AND USES THEREOF

(71) Applicant: Bicycle Tx Limited, Cambridge (GB)

(72) Inventors: Gavin BENNETT, Cambridge (GB); Lisa MAHNKE, Boston, MA (US)

(21) Appl. No.: 17/779,226

(22) PCT Filed: Nov. 27, 2020
(86) PCT No.: PCT/GB2020/053026

§ 371 Date: May 24, 2022

Related U.S. Application Data
(60) Provisional application No. 62/940,966, filed on Nov. 27, 2019.

(51) Int. Cl. A61K 47/18; A61K 47/26; A61K 9/19; A61K 35/00; A61K 9/00

(52) U.S. Cl. CPC A61K 47/183; A61K 47/26; A61K 9/19; A61K 35/00; A61K 9/0019

(57) ABSTRACT

The present invention relates to a Bicycle toxin conjugate BT5528, or pharmaceutically acceptable salts thereof, or pharmaceutical compositions thereof, and uses thereof.

### CROSS-REFERENCE TO RELATED APPLICATIONS

The present application claims priority to U.S. provisional patent application Ser. No. 62/940,966, filed Nov. 27, 2019, the entirety of which is incorporated herein by reference.

### TECHNICAL FIELD OF THE INVENTION

The present invention relates to Bicycle toxin conjugates, or pharmaceutically acceptable salts thereof, or pharmaceutical compositions thereof. The present invention also provides uses of Bicycle toxin conjugates, or pharmaceutically acceptable salts thereof, or pharmaceutical compositions thereof, for preventing or treating a disease, disorder, or condition characterized by overexpression of EphA2 in diseased tissue.

### BACKGROUND OF THE INVENTION

Cyclic peptides are able to bind with high affinity and target specificity to protein targets and hence are an attractive molecule class for the development of therapeutics. In fact, several cyclic peptides are already successfully used in the clinic, as for example the antibacterial peptide vancomycin, the immunosuppressant drug cyclosporine or the anti-cancer drug octreotide (Driggers et al. (2008), Nat Rev Drug Discov 7 (7), 608-24). Good binding properties result from a relatively large interaction surface formed between the peptide and the target as well as the reduced conformational flexibility of the cyclic structures. Typically, macrocycles bind to surfaces of several hundred square angstrom, as for example the cyclic peptide CXCR4 antagonist CVX15 (400 Å²; Wu et al. (2007), Science 330, 1066-71), a cyclic peptide with the Arg-Gly-Asp motif binding to integrin αVb3 (355 Å²) (Xiong et al. (2002), Science 296 (5565), 151-5) or the cyclic peptide inhibitor upain-1 binding to urokinase-type plasminogen activator (603 Å²; Zhao et al. (2007), J Struct Biol 160 (1), 1-10).

Due to their cyclic configuration, peptide macrocycles are less flexible than linear peptides, leading to a smaller loss of entropy upon binding to targets and resulting in a higher binding affinity. The reduced flexibility also leads to locking target-specific conformations, increasing binding specificity compared to linear peptides. This effect has been exemplified by a potent and selective inhibitor of matrix metalloproteinase 8, (MMP-8) which lost its selectivity over other MMPs when its ring was opened (Cherney et al. (1998), J Med Chem 41 (11), 1749-51). The favorable binding properties achieved through macrocyclization are even more pronounced in multicyclic peptides having more than one peptide ring as for example in vancomycin, nisin and actinomycin.

Different research teams have previously tethered polypeptides with cysteine residues to a synthetic molecular structure (Kemp and McNamara (1985), J. Org. Chem; Timmerman et al. (2005), ChemBioChem). Meloen and co-workers had used tris(bromomethyl)benzene and related molecules for rapid and quantitative cyclisation of multiple peptide loops onto synthetic scaffolds for structural mimicry of protein surfaces (Timmerman et al. (2005), ChemBioChem). Methods for the generation of candidate drug compounds wherein said compounds are generated by linking cysteine containing polypeptides to a molecular scaffold as for example TATA (1,1′,1″-(1,3,5-triazinane-1,3,5-triyl)triprop-2-en-1-one, Heinis et al. Angew Chem, Int Ed. 2014; 53:1602-1606).

Phage display-based combinatorial approaches have been developed to generate and screen large libraries of bicyclic peptides to targets of interest (Heinis et al. (2009), Nat Chem Biol 5 (7), 502-7 and WO 2009/098450). Briefly, combinatorial libraries of linear peptides containing three cysteine residues and two regions of six random amino acids (Cys-(Xaa)₆-Cys-(Xaa)₆-Cys) were displayed on phage and cyclised by covalently linking the cysteine side chains to a small molecule scaffold.

### SUMMARY OF THE INVENTION

According to one aspect, the invention provides a pharmaceutical composition comprising BT5528, or a pharmaceutically acceptable salt thereof, histidine hydrochloride, sucrose, and Polysorbate 20. In some embodiments, a pharmaceutical composition comprising BT5528, or a pharmaceutically acceptable salt thereof, histidine hydrochloride, sucrose, and Polysorbate 20 is a lyophilized powder.

In another aspect, the invention provides a pharmaceutical composition comprising BT5528, or a pharmaceutically acceptable salt thereof, histidine hydrochloride, sucrose, Polysorbate 20, and dextrose. In some embodiments, a pharmaceutical composition comprising BT5528, or a pharmaceutically acceptable salt thereof, histidine hydrochloride, sucrose, Polysorbate 20, and dextrose is a pharmaceutical formulation in water.

In another aspect, the invention provides a method for treating advanced malignancies associated with EphA2 expression in a patient comprising administering to the patient a pharmaceutical composition as described herein. In some embodiments, the invention provides a method for treating advanced malignancies associated with EphA2 expression in a patient comprising administering to the patient weekly by IV infusion a pharmaceutical formulation comprising BT5528, or a pharmaceutically acceptable salt thereof, histidine, sucrose, Polysorbate 20, and dextrose in water.

### DETAILED DESCRIPTION OF CERTAIN EMBODIMENTS

### 1. General Description of Certain Embodiments of the Invention

Stable lyophilized formulations have been developed for BT5528, which can be reconstituted for administration. During the development process, it was observed that BT5528 adsorbed to the surface of the vials and that reconstitution was challenging. The cause of adsorption of BT5528 to the surface of the vials was investigated during separate lyophilization cycles.

A number of hypotheses were presented as potential causes, high sodium chloride concentration in the final product, a more basic pH of the reconstituted product, silanised vials and over-drying of the peptide. Each of these hypotheses were assessed together with a formulation screen of alternative sugars, surfactant and a lower BT5528 concentration to investigate if this improved the reconstitution characteristics. An improvement in the reconstitution time was obtained with a lower pH pre-lyophilization, however, oily droplets remained on the surface of the vial. A reduction in the sodium chloride content, use of silanised vials and a lower secondary drying temperature (to increase the final moisture content of the product) did not improve the reconstitution characteristics. Alternative sugars or surfactant did not generate an improvement.

Surprisingly, it was found that a reduction in BT5528 concentration from 4 mg/mL to 2 mg/mL was found to improve the reconstitution characteristics. The reconstitution time was reduced and no oily droplets were observed on the surface of the vials post-reconstitution. The recovery vs theoretical was at target in the 2 mg/mL formulation compared to consistently below target for the 4 mg/mL formulations.

Accordingly, in one aspect, the invention provides a solid pharmaceutical composition comprising BT5528, or a pharmaceutically acceptable salt thereof, which is prepared by removing solvent(s), for example, by lyophilization, from a liquid formulation wherein the concentration of BT5528, or a pharmaceutically acceptable salt thereof, is about 2-4 mg/mL.

In another aspect, the invention provides methods of using a pharmaceutical composition described herein for treating an advanced solid tumor malignancy associated with EphA2-expression.

### 2. Compounds and Definitions

The term “BT5528,” as used herein, is a Bicycle toxin conjugate having a structure as shown below, wherein the molecular scaffold is 1,1′,1″-(1,3,5-triazinane-1,3,5-triyl)triprop-2-en-1-one (TATA), and the peptide ligand comprises the amino acid sequence:

(SEQ ID NO: 1)
(β-Ala)-Sar₁₀-A(HArg)D-Cᵢ(HyP)LVNPLCᵢᵢLHP
(D-Asp)W(HArg)Cᵢᵢᵢ

wherein Sar is sarcosine, HArg is homoarginine, and HyP is hydroxyproline.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

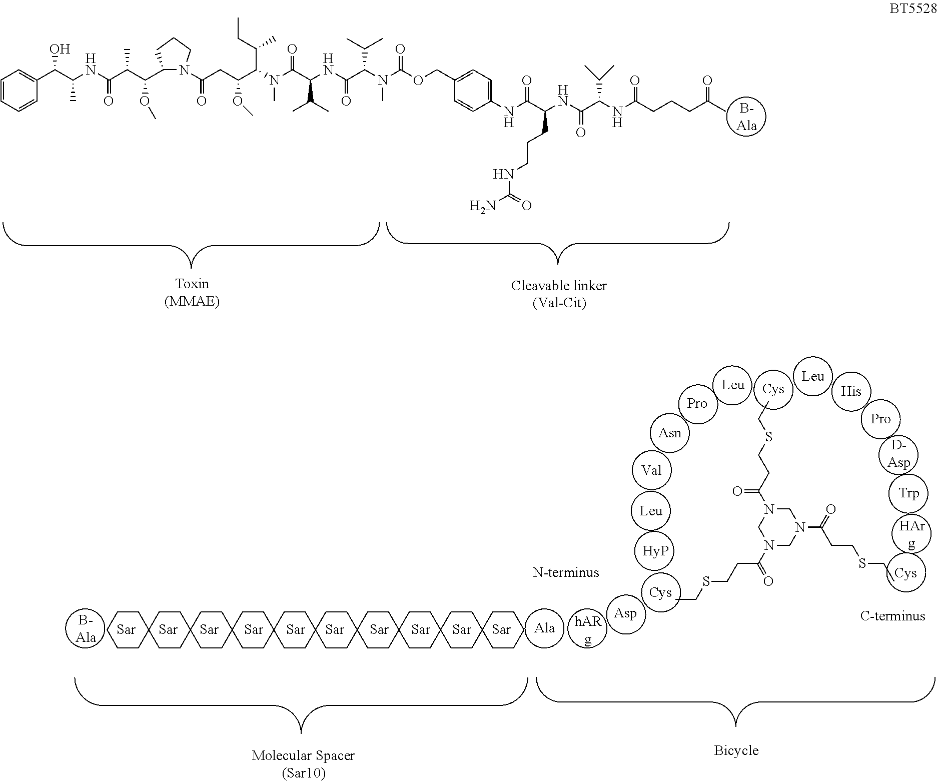

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. It will be appreciated that salt forms are within the scope of this invention, and references to peptide ligands include the salt forms of said ligands.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two.

As used herein, the term “about” shall have the meaning of within 10% of a given value or range. In some embodiments, the term “about” refers to within 10%, 9%, 8%, 7%, 6%, 5%, 4%, 3%, 2%, or 1% of a given value.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

### 3. Pharmaceutical Compositions

According to one aspect, the invention provides a pharmaceutical composition comprising BT5528, or a pharmaceutically acceptable salt thereof, and a pharmaceutically acceptable excipient or carrier. In some embodiments, a pharmaceutical composition of the invention comprises about 21.2 mg BT5528, or a pharmaceutically acceptable salt thereof.

In some embodiments, a pharmaceutical composition of the invention is a solid pharmaceutical composition. In some embodiments, a solid pharmaceutical composition of the invention is powders. In some embodiments, a pharmaceutical composition of the invention is lyophilized powder. In some embodiments, a solid pharmaceutical composition of the invention is granules.

In some embodiments, a pharmaceutical composition of the invention is a liquid pharmaceutical composition. In some embodiments, a liquid pharmaceutical composition of the invention is a pharmaceutical formulation in an acceptable vehicle or solvent. In some embodiments, an acceptable vehicle or solvent is selected from sterile water, Ringer's solution, U.S.P. and isotonic sodium chloride solution. In some embodiments, an acceptable vehicle or solvent is sterile water. In some embodiments, an acceptable vehicle or solvent is a sterile injectable medium. In some embodiments, a liquid pharmaceutical composition of the invention comprises about 2-4 mg/mL BT5528, or a pharmaceutically acceptable salt thereof.

In some embodiments, a pharmaceutically acceptable excipient or carrier comprises a buffering agent. In some embodiments, a buffering agent is selected from phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. In some embodiments, a buffering agent is histidine hydrochloride. In some embodiments, a buffering agent is sodium hydroxide. In some embodiments, a buffering agent is hydrochloric acid.

In some embodiments, a buffering agent is at an amount to adjust pH of a pharmaceutical composition of the invention to about 6-8. In some embodiments, a buffering agent is histidine hydrochloride at an amount of about 1-3 mg per mg of BT5528, or a pharmaceutically acceptable thereof. In some embodiments, histidine hydrochloride is at an amount of about 1.31 or 2.62 mg per mg of BT5528, or a pharmaceutically acceptable thereof. In some embodiments, a liquid pharmaceutical composition of the invention comprises histidine hydrochloride at a concentration of about 5.24 mg/mL.

In some embodiments, a liquid pharmaceutical composition of the invention is at a pH of about 6-8. In some embodiments, a liquid pharmaceutical composition of the invention is at a pH of about 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0. In some embodiments, a liquid pharmaceutical composition of the invention is at a pH of about 6.5 or 7.0.

In some embodiments, a pharmaceutically acceptable excipient or carrier comprises an inert pharmaceutically acceptable excipient or carrier. In some embodiments, an inert pharmaceutically acceptable excipient or carrier is sodium citrate or dicalcium phosphate. In some embodiments, an inert pharmaceutically acceptable excipient or carrier is a filler or extender. In some embodiments, a filler or extender is starches, lactose, sucrose, glucose, mannitol, or silicic acid. In some embodiments, an inert pharmaceutically acceptable excipient or carrier is a binder. In some embodiments, a binder is carboxymethylcellulose, alginates, gelatin, polyvinylpyrrolidinone, sucrose, or acacia. In some embodiments, an inert pharmaceutically acceptable excipient or carrier is selected from sucrose, trehalose, dextrose, or a combination thereof. In some embodiments, an inert pharmaceutically acceptable excipient or carrier is sucrose.

In some embodiments, an inert pharmaceutically acceptable excipient or carrier (e.g., sucrose) is at an amount of about 10-35 mg per mg of BT5528, or a pharmaceutically acceptable thereof. In some embodiments, an inert pharmaceutically acceptable excipient or carrier (e.g., sucrose) is at an amount of about 15 or 30 mg per mg of BT5528, or a pharmaceutically acceptable thereof. In some embodiments, a liquid pharmaceutical composition comprises an inert pharmaceutically acceptable excipient or carrier (e.g., sucrose) at a concentration of about 60 mg/mL.

In some embodiments, a pharmaceutically acceptable excipient or carrier comprises a surfactant. In some embodiments, a surfactant is a polysorbate (e.g., polysorbate-20, polysorbate-40, polysorbate-60, polysorbate-65, polysorbate-80, polysorbate-85, or a combination thereof). In some embodiments, a surfactant is selected from poloxamers (e.g., poloxamer 188); Triton™; sodium dodecyl sulfate (SDS); sodium laurel sulfate; sodium octyl glycoside; lauryl-sulfobetaine, myristyl-sulfobetaine, linoleyl-sulfobetaine, stearyl-sulfobetaine, lauryl-sarcosine, myristyl-sarcosine, linoleyl-sarcosine, stearyl-sarcosine, linoleyl-betaine, myristyl-betaine, cetyl-betaine, lauroamidopropyl-betaine, cocamidopropyl-betaine, linoleamidopropyl-betaine, myristamidopropyl-betaine, palmidopropyl-betaine, isostearamidopropyl-betaine (e.g. lauroamidopropyl), myristarnidopropyl-, palmidopropyl-, or isostearamidopropyl-dimethylamine; sodium methyl cocoyl-, or disodium methyl ofeyl-taurate; and the Monaquat™ series (Mona Industries, Inc., Paterson, N.J.), poly ethyl glycol, polyp ropyl glycol, and copolymers of ethylene and propylene glycol (e.g. pluronics, PF68). In some embodiments, a surfactant is Polysorbate 20.

In some embodiments, a surfactant (e.g., Polysorbate 20) is at an amount of about 0.01-0.15 mg per mg of BT5528, or a pharmaceutically acceptable thereof. In some embodiments, a surfactant (e.g., Polysorbate 20) is at an amount of about 0.025, 0.05, or 0.1 mg per mg of BT5528, or a pharmaceutically acceptable thereof. In some embodiments, a liquid pharmaceutical composition comprises a surfactant (e.g., Polysorbate 20) at a concentration of about 0.1 or 0.2 mg/mL.

In some embodiments, a pharmaceutically acceptable excipient or carrier comprises an isotonicity adjusting agent. In some embodiments, an isotonicity adjusting agent is sodium chloride, dextrose, calcium chloride, or a combination thereof. In some embodiments, an isotonicity adjusting agent is dextrose. In some embodiments, an isotonicity adjusting agent is sodium chloride. In some embodiments, an isotonicity adjusting agent is a combination of sodium chloride and dextrose.

In some embodiments, the invention provides a pharmaceutical composition comprising BT5528, or a pharmaceutically acceptable salt thereof, histidine hydrochloride, sucrose, and Polysorbate 20. In some embodiments, a pharmaceutical composition of the invention comprises:
BT5528, or a pharmaceutically acceptable salt thereof;
about 1.31-2.62 mg histidine hydrochloride per mg of BT5528, or a pharmaceutically acceptable thereof;
about 15-30 mg sucrose per mg of BT5528, or a pharmaceutically acceptable thereof; and
about 0.05-0.1 mg Polysorbate 20 per mg of BT5528, or a pharmaceutically acceptable thereof.

In some embodiments, the invention provides a solid pharmaceutical composition, which is a lyophilized powder, comprising:
about 21.2 mg BT5528, or a pharmaceutically acceptable salt thereof;
about 55.5 mg histidine hydrochloride per mg of BT5528, or a pharmaceutically acceptable thereof;
about 636 mg sucrose per mg of BT5528, or a pharmaceutically acceptable thereof; and
about 1.06-2.12 mg Polysorbate 20 per mg of BT5528, or a pharmaceutically acceptable thereof.

In some embodiments, the invention provides a solid pharmaceutical composition, which is a lyophilized powder, comprising:
about 21.2 mg BT5528, or a pharmaceutically acceptable salt thereof;
about 27.8 mg histidine hydrochloride per mg of BT5528, or a pharmaceutically acceptable thereof;
about 318 mg sucrose per mg of BT5528, or a pharmaceutically acceptable thereof; and
about 1.06 mg Polysorbate 20 per mg of BT5528, or a pharmaceutically acceptable thereof.

In some embodiments, the invention provides a liquid pharmaceutical composition comprising:
about 2-4 mg/mL BT5528, or a pharmaceutically acceptable salt thereof;
about 5.25 mg/mL histidine hydrochloride;
about 60 mg/mL sucrose; and
about 0.1-0.2 mg/mL Polysorbate 20.

In some embodiments, the invention provides a liquid pharmaceutical composition prepared by dissolving a solid pharmaceutical composition of the invention in water. In some embodiments, the invention provides a liquid pharmaceutical composition prepared by dissolving a solid pharmaceutical composition of the invention in an injectable medium (e.g., saline or 5% dextrose). In some embodiments, the invention provides a liquid pharmaceutical composition prepared by reconstitute a solid pharmaceutical composition of the invention in water, followed by dilution with 5% dextrose. In some embodiments, a liquid pharmaceutical composition is diluted into a 5% dextrose IV bag for IV administration. In some embodiments, a liquid pharmaceutical composition in a 5% dextrose IV bag is stored under room temperature (about 20-25° C.) for up to about 4 hours before IV administration. In some embodiments, a liquid pharmaceutical composition in a 5% dextrose IV bag is stored under refrigerated (about 2-8° C.) conditions for up to about 20 hours before IV administration. In some embodiments, a liquid pharmaceutical composition in a 5% dextrose IV bag is stored under refrigerated (about 2-8° C.) conditions for up to about 20 hours, followed by storage under room temperature (about 20-25° C.) for up to about 4 hours, before IV administration.

In some embodiments, the invention provides a solid pharmaceutical composition comprising BT5528, or a pharmaceutically acceptable salt thereof, histidine hydrochloride, sucrose, Polysorbate 20, and dextrose. In some embodiments, the invention provides a liquid pharmaceutical composition comprising BT5528, or a pharmaceutically acceptable salt thereof, histidine hydrochloride, sucrose, Polysorbate 20, dextrose, and water. In some embodiments, the components of the pharmaceutical compositions are at the amount, concentration, and ratio as described above.

### 4. Uses of the Pharmaceutical Compositions

In one aspect, the invention provides a method, or a use, for treating an advanced solid tumor malignancy associated with EphA2-expression in a patient comprising administering to the patient a pharmaceutical composition as described herein. In some embodiments, an advanced solid tumor malignancy associated with EphA2-expression is selected from non-small-cell lung cancer (NSCLC), ovarian cancer, triple-negative breast cancer (TNBC), gastric/upper gastrointestinal (GI), pancreatic and urothelial cancers. In some embodiments, an advanced solid tumor malignancy associated with EphA2-expression is an adenocarcinoma subtype of NSCLC (adeno-NSCLC).

In some embodiments, a method of the invention comprises administering to a patient intravenously a pharmaceutical composition as described herein. In some embodiments, a pharmaceutical composition of the invention is administered by an IV injection. In some embodiments, a pharmaceutical composition of the invention is administered by an IV infusion. In some embodiments, an IV infusion of a pharmaceutical composition of the invention lasts about 5-30 minutes. In some embodiments, an IV infusion of a pharmaceutical composition of the invention lasts about 30-90 minutes. In some embodiments, an IV infusion of a pharmaceutical composition of the invention lasts about 30, 35, 40, 45, 50, 55, 60, 65, 70, 75, 80, 85, or 90 minutes. In some embodiments, an IV infusion of a pharmaceutical composition of the invention lasts about 60 minutes. In some embodiments, an IV infusion of a pharmaceutical composition of the invention lasts about 2, 2.5, 3, 3.5, or 4 hours.

In some embodiments, a pharmaceutical composition of the invention is administered to a patient once every 1, 2, 3, 4, 5, 6, or 7 days. In some embodiments, a pharmaceutical composition of the invention is administered to a patient weekly. In some embodiments, a pharmaceutical composition of the invention is administered to a patient once every two weeks.

In some embodiments, a pharmaceutical composition of the invention is administered at a dose of about 1-27 mg/m². In some embodiments, a pharmaceutical composition of the invention is administered at a dose of about 2-20 mg/m². In some embodiments, a pharmaceutical composition of the invention is administered at a dose of about 2-20 mg/m². In some embodiments, a pharmaceutical composition of the invention is administered at a dose of about 2.2, 4.4, 7.3, 11, 14.6, or 19.4 mg/m². In some embodiments, a pharmaceutical composition of the invention is administered at a dose of about 1.5-3.5, 3.5-5.5, 6.5-8.5, 10-12, 13.5-15.5, or 18.5-20.5 mg/m². In some embodiments, a pharmaceutical composition of the invention is administered at a dose of about 1-10 or 10-20 mg/m². In some embodiments, a pharmaceutical composition of the invention is administered at a dose of about 21, 22, 23, 24, 25, 26, or 27 mg/m².

In some embodiments, a pharmaceutical composition of the invention is administered to a patient at least 18 years-of-age.

In some embodiments, a pharmaceutical composition of the invention is administered to a patient having an Eastern Cooperative Oncology Group (ECOG) Performance Status score of 0 or 1. The ECOG Performance Status scores of 0 and 1 are described in Example 1.

In some embodiments, a pharmaceutical composition of the invention is administered to a patient having measurable disease per Response Evaluation Criteria in Solid Tumors (RECIST) v1.1.

In some embodiments, a pharmaceutical composition of the invention is administered to a patient having acceptable organ function. In some embodiments, a patient having acceptable organ function has laboratory data selected from the following:
Renal function: creatinine clearance of ≥50 mL/min by the Cockcroft-Gault equation or as measured by 24-hour urine collection;
Total bilirubin ≤1.5×ULN (upper limit of normal);
Serum albumin ≥2.5 g/dL;
Aspartate aminotransferase (AST) ≤2.5×ULN or ≤5×ULN in the presence of liver metastases;
Alanine aminotransferase (ALT) ≤2.5×ULN or ≤5×ULN in the presence of liver metastases; and
International normal ratio (INR) <1.3 or ≤institutional ULN (anticoagulants not allowed).

In some embodiments, a pharmaceutical composition of the invention is administered to a patient having acceptable hematologic function. In some embodiments, a patient having acceptable hematologic function has laboratory data selected from the following:
Hemoglobin ≥9 g/dL;
Absolute neutrophil count (ANC) ≥1500 cells/mm³; and
Platelet count ≥75,000 cells/mm³.

In some embodiments, a patient has no red blood cell or platelet transfusions or growth factors within 4 weeks of the first dose of a pharmaceutical composition of the invention.

In some embodiments, a patient has an adenocarcinoma subtype of NSCLC (adeno-NSCLC), who has exhausted all standard treatment options including progression on or after platinum-based chemotherapy, and/or has failed at least one prior line of therapy with evidence of radiographic progression on the most recent line of therapy. In some embodiments, a patient has EGFR, ALK, NTRK, ROS1 or other genomic tumor aberrations. In some embodiments, a patient has not received appropriate treatment for driver mutation disease. In some embodiments, a patient has not received immunotherapy at least 28 days prior to the first dose of a pharmaceutical composition of the invention.

In some embodiments, a patient has not had a chemotherapy treatment within 14 days prior to first dose of a pharmaceutical composition of the invention. In some embodiments, a patient has not had an anticancer treatment within 28 days or 5 half-lives, whichever shorter, prior to first dose of a pharmaceutical composition of the invention. In some embodiments, a patient has prior toxicities which have resolved to grade 1 per Common Terminology Criteria for Adverse Events (CTCAE) v 5.0 (except alopecia which must be no greater than Grade 2).

In some embodiments, a patient has not had an experimental treatment within 4 weeks of first dose of a pharmaceutical composition of the invention.

In some embodiments, a patient does not have a current treatment with strong inhibitors or inducers of CYP3A4 or strong inhibitors of P-gp including herbal- or food-based.

In some embodiments, a patient does not have any sensitivity to any of the ingredients of a pharmaceutical composition of the invention, or to monomethyl auristatin E (MMAE).

In some embodiments, a patient does not have any significant medical condition, life-threatening illness, active uncontrolled infection or organ system dysfunction (such as ascites, coagulopathy, encephalopathy).

In some embodiments, a patient has not had any major surgery (excluding placement of vascular access) within 4 weeks of first dose of a pharmaceutical composition of the invention.

In some embodiments, a patient has not received a live vaccine within 30 days of first dose of a pharmaceutical composition of the invention.

In some embodiments, a patient does not have uncontrolled, symptomatic brain metastases. In some embodiments, a patient has stable neurologic status following local therapy for at least 4 weeks without using steroids or on stable or decreasing dose of less than or equal to 10 mg daily prednisone or equivalent.

In some embodiments, a patient does not have uncontrolled hypertension (systolic blood pressure [BP]≥139 mmHg; diastolic BP≥89 mmHg) prior to first dose of a pharmaceutical composition of the invention. In some embodiments, a patient has hypertension which has been in stable control for at least 3 months prior to first dose of a pharmaceutical composition of the invention.

In some embodiments, a patient does not have a history of a cerebral vascular event (stroke or transient ischemic attack), unstable angina, myocardial infarction, congestive heart failure or symptoms of New York Heart Association Class III-IV documented within 6 months prior to first dose of a pharmaceutical composition of the invention.

In some embodiments, a patient does not have mean resting corrected QT interval (QTcF) >470 msec within 6 months prior to first dose of a pharmaceutical composition of the invention.

In some embodiments, a patient does not have, within 6 months prior to first dose of a pharmaceutical composition of the invention, any factors that increase the risk of QTc prolongation or risk of arrhythmic events such as heart failure, hypokalemia, congenital long QT syndrome, family history of long QT syndrome or unexplained sudden death under 40 years-of-age, or any concomitant medication known to prolong the QT interval.

In some embodiments, a patient does not have, within 6 months prior to first dose of a pharmaceutical composition of the invention, any clinically important abnormalities in rhythm, conduction, or morphology of resting electrocardiograms (ECGs), e.g., complete left bundle branch block, third degree heart block.

In some embodiments, a patient does not have human immunodeficiency virus (HIV) or acquired immune deficiency syndrome (AIDS).

In some embodiments, a patient does not have a positive hepatitis B surface antigen and/or anti-hepatitis B core antibody.

In some embodiments, a patient has a negative polymerase chain reaction (PCR) assay and has an appropriate antiviral therapy.

In some embodiments, a patient has an active hepatitis C infection with positive viral load if hepatitis C virus (HCV) antibody positive.

In some embodiments, a patient has been treated for hepatitis C infection and has sustained virologic response of ≥12 weeks.

In some embodiments, a patient does not have thromboembolic events and/or bleeding disorders within 3 months (e.g., deep vein thrombosis [DVT] or pulmonary embolism [PE]) prior to the first dose of a pharmaceutical composition of the invention.

In some embodiments, a patient does not have another malignancy within 3 years before the first dose of a pharmaceutical composition of the invention. In some embodiments, a patient does not have any residual disease from a previously diagnosed malignancy (excluding adequately treated with curative intent basal cell carcinoma, squamous cell of the skin, cervical intraepithelial neoplasia/cervical carcinoma in situ or melanoma in situ or ductal carcinoma in situ of the breast).

In some embodiments, a patient does not have systemic anti-infective treatment or fever within the last 14 days prior to first dose of a pharmaceutical composition of the invention.

In some embodiments, the invention provides a combination use of a pharmaceutical composition of the invention and Nivolumab, for treating an advanced solid tumor malignancy associated with EphA2-expression. Nivolumab can be administered as described on the label, which can be found at https://www.opdivohcp.com/dosing/dosing-schedules, the content of which is incorporated herein by reference in its entirety. In some embodiments, Nivolumab is administered 240 mg every 2 weeks. In some embodiments, Nivolumab is administered 480 mg every 4 weeks. In some embodiments, Nivolumab is administered as a 30-minute IV infusion.

In some embodiments of a combination use, a patient is not previously known being intolerance to an immune checkpoint inhibitor. In some embodiments of a combination use, a patient is not known being hypersensitivity to checkpoint inhibitor therapy. In some embodiments of a combination use, a patient has no prior organ transplant. In some embodiments of a combination use, a patient is not previously diagnosed with clinically relevant immunodeficiency. In some embodiments of a combination use, a patient does not have active systemic infection requiring therapy. In some embodiments of a combination use, a patient does not take more than 10 mg daily prednisone equivalent or other strong immunosuppressant. In some embodiments of a combination use, a patient does not have a history of autoimmune disease except alopecia or vitiligo. In some embodiments of a combination use, a patient does not have a history of interstitial lung disease.

### EXEMPLIFICATION

The following examples are intended to illustrate the invention and are not to be construed as being limitations thereon. All amino acids, unless noted otherwise, were used in the L-configurations.

### Example 1. Preparation of BT5528 Lyophilized Drug Product

A process assessment was first performed, followed by a thermal assessment of the candidate formulation. A test lyophilization with associated stability was undertaken followed by lyophilization cycle optimization, additional formulation screening and a filtration assessment.

### 1. BT5528 Process Assessment and Freeze/Thaw Stability

The bulk solution appearance, pH, density and BT5528 assay/related substances were recorded. Additional samples were subjected to three freeze (−20° C.)/thaw cycles, assessing appearance, pH, UPLC and sub-visible particulates (MFI) after each cycle.

### 1.1. Solution Preparation

14 g of WFI (70% w/v) was weighed into a small glass beaker, to this 104 mg of Histidine HCl was weighed out and added with rinsing then stirred until dissolved. Once dissolved 1.2 g of sucrose was weighed out, added then stirred magnetically until dissolved. The pH of the solution was measured and adjusted to target (pH 7.7-8.1). The initial pH was 3.42 pH and was adjusted to 8.07 pH using 0.1M NaOH solution.

A 1% (w/v) Polysorbate 20 solution was prepared by dissolving 0.5 g of Polysorbate 20 in 50 mL of WFI. 400 μL of the 1% (w/v) Polysorbate 20 solution was added to the Histidine/Sucrose solution while stirring. 86.8 mg of BT5528 was weighed out and added slowly to the solution with continuous stirring. Complete dissolution of the API took approximately 90 minutes.

The pH of the solution was measured and adjusted to target (pH 6.8-7.2). The pH was 7.79 pH after the addition of API and was adjusted to 7.19 pH using 0.1 M HCl solution. The solution was transferred to a 20 mL volumetric flask and make to final volume with WFI. The solution was filtered through a single 0.22 μm, PES membrane syringe filter. The final solution was a clear, colourless solution which was free from any visible particulates. The density of the final solution was measured, 1.025 g/cm³. A pre- and post-filtration sample was passed to analytical for HPLC analysis. The remaining bulk solution was filled in 2 mL volumes into 2 mL glass vials and subjected to three, freeze/thaw cycles.

### 1.2. Results

### 1.2.1. Appearance and pH

There was no significant change to the appearance or pH over the course of the three freeze/thaw cycles.

TABLE 1-1
Solution appearance and pH

| Time-point/storage | Appearance | pH |
|---|---|---|
| Pre Filtration | Clear, colourless solution free | 7.2 |
| | from visible particulates | |
| Post Filtration | Clear, colourless solution free | N/A |
| | from visible particulates | |
| Freeze Thaw | Clear, colourless solution free | 7.3 |
| Cycle 1 | from visible particulates | |
| Freeze Thaw | Clear, colourless solution free | 7.3 |
| Cycle 2 | from visible particulates | |
| Freeze Thaw | Clear, colourless solution free | 7.3 |
| Cycle 3 | from visible particulates | |

### 1.2.2. Assay and Related Substances

The results show there was no change in assay or related substances during pre- and postfiltration. There was also no change in assay or related substances during the freeze/thaw assessment.

TABLE 1-2
Assay

| | Vial 1 Content | Vial 2 Content | Average Content | Rec./theory¹ | Rec./Pre² |
|---|---|---|---|---|---|
| Time-point/storage | (mg/mL) | (mg/mL) | (mg/mL) | (%) | (%) |
| Pre Filtration | 3.8259 | 3.8287 | 3.83 | 95.68 | — |
| Post Filtration | 3.8249 | 3.8305 | 3.83 | 95.69 | 100.01 |
| Freeze Thaw Cycle 1 | 3.8189 | 3.8102 | 3.81 | 95.36 | 99.67 |
| Freeze Thaw Cycle 2 | 3.8021 | 3.8316 | 3.82 | 95.42 | 99.73 |
| Freeze Thaw Cycle 3 | 3.8297 | 3.8241 | 3.83 | 95.67 | 99.99 |

¹As percentage of theoretical 4 mg/mL.
²As percentage of pre filtration result.

TABLE 1-3
Related substances

| | Sample and amount (% area) | | | | |
|---|---|---|---|---|---|
| | | | Freeze | Freeze | Freeze |
| | Pre | Post | Thaw | Thaw | Thaw |
| RRT | Filtration | Filtration | Cycle 1 | Cycle 2 | Cycle 3 |
| 0.97 | 0.12 | 0.13 | 0.11 | 0.11 | 0.11 |
| 0.99 | 2.20 | 2.18 | 2.18 | 2.19 | 2.16 |
| 1.03 | 0.98 | 0.97 | 0.97 | 0.94 | 1.00 |
| 1.05 | 0.21 | 0.22 | 0.17 | 0.16 | 0.20 |
| 1.12 | 0.10 | <LOQ | <LOQ | 0.10 | <LOQ |
| 1.15 | 0.12 | 0.12 | 0.14 | 0.13 | 0.13 |
| Total¹ | 3.73 | 3.62 | 3.57 | 3.63 | 3.60 |

¹Sum of related substances ≥0.10%

### 1.3. Discussion and Conclusions

There was no loss of assay during filtration indicating no adsorption of the API to a PES membrane. The assay and related substances data did not change after vials were subjected to three freeze/thaw cycles demonstrating the API to be stable during freeze/thaw cycles.

### 2. Stage 3—Thermal Assessment

The thermal characteristics of the BT5528 formulation was assessed by freeze drying microscopy (FDM) and differential scanning calorimetry (DSC) to identify any collapse temperature or thermal events which would influence the design of the lyophilization cycle.

The formulation details are shown in Table 1-4. By assessing the thermal characteristics of the active and placebo formulations the parameters for a freeze drying cycle could be determined.

TABLE 1-4
Formulation composition

| | Concentration |
|---|---|
| Component | (mg/mL) |
| BT5528 | 4.0 |
| Histidine HCl | 5.24 |
| Sucrose | 60.0 |
| Polysorbate 20 | 0.2 |
| Sodium hydroxide | q.s. to target pH |
| WFI | q.s. to 1 mL |

### 2.1. Freeze Drying Microscope (FDM)

FDM was used to determine the collapse temperature of the formulation. Several samples were analyzed to refine the FDM cycle set point parameters. Each analysis was performed using a 10× objective lens with bright field transmitted light. The key observations from each analysis performed are shown in Table 1-5.

TABLE 1-5
Thermal observations by FDM

| | Freezing | Onset of collapse | Collapse |
|---|---|---|---|
| Run | (° C.) | (° C.) | (° C.) |
| 1 | −29.5 | −32.7 | −30.2 |
| 2 | −28.2 | −32.5 | −29.3 |
| 3 | −28.2 | −31.7 | −30.2 |
| 4 | −28.2 | −31.9 | −30.4 |

Subtle changes in sample structure, e.g. ill-defined drying front, were noted as the first indications of sample collapse, followed by the appearance of bright spots. These visual observations were interpreted as the onset of collapse. Further loss of structure was evident as larger bright regions developed which spread across the sample. These regions subsequently formed a rip, resulting in gross collapse. This development of inter-connected bright regions/rips was taken as collapse of the product.

### 2.2. Differential Scanning Calorimetry (DSC)

In addition to FDM, the formulation were analyzed by DSC using the parameters displayed in Table 1-6. A summary of the key observations is shown in Table 1-7.

TABLE 1-6
DSC set point parameters for no annealing

| Parameter | Setting |
|---|---|
| Cycle | 25° C. to −50° C. at 1° C./min, |
| | hold 10 mins at −50° C., −50° C. |
| | to 25° C. at 1° C./min |
| Crucible | Sealed 100 μL aluminium |
| Reference sample | Empty crucible |
| Purge gas | 60 mL/min N₂ |
| Dry gas | 170 mL/min N₂ |

TABLE 1-7
Key observations from DSC thermograms

| | |
|---|---|
| | Temperature |
| Observation | (° C.) |

| | | |
|---|---|---|
| Freezing onset | −9.69 | −18.74 |
| Glass transition/eutectic melt | None detected | None detected |
| Melting onset | −13.21 | −10.72 |

DSC showed large, well-defined freezing exotherms and melting endotherms. There were no other notable thermal events observed.

### 2.2.1. Discussion and Conclusions

The data generated by FDM and DSC was used to aid the design of a lyophilization cycle. These temperatures gave an indication of the freezing temperature and highest permissible product temperature during primary drying. The collapse temperature of the formulation was approximately −30° C., this temperature will determine the primary drying temperature of the lyophilization cycle.

Based on the results of the thermal assessment, the lyophilization cycle would aim to maintain the product temperature at 5-10° C. below the collapse temperature during primary drying, which in this case is approximately −30° C.

### 3. Stage 5—Test Lyophilization

The thermal characteristics of the BT5528 formulation were determined in Stage 4—thermal assessment. The thermal data generated in Stage 4 showed a collapse temperature of approximately −30° C.

Based on this data, the test lyophilization cycle detailed in Table 1-8 was designed to maintain the product temperature at 5-10° C. below the collapse temperature. Sufficient vials were prepared to support the stability study of the lyophilized product as detailed in Table 1-9 and a reconstitution stability assessment. The solution at 4 mg/mL was filled at a volume of 1 mL into 2 mL clear Type I glass vials. The lyophilized product would be reconstituted with 1 mL of WFI to achieve the target concentration of 4 mg/mL. The area immediately surrounding the active vials was packed out with vials containing 1 mL of buffer solution to more closely mimic the conditions of a full chamber.

### 3.1. Solution Preparation

˜80 mL of WFI (˜70% w/v final volume) was added to a glass beaker to which 628.8 mg of histidine HCl was added and stirred magnetically until dissolved. Once dissolved, 7.2 g of sucrose was weighed out and added to the WFI/histidine solution then stirred until dissolved. The pH of the solution was measured and adjusted to target (pH 7.7-8.1) with 0.1 M sodium hydroxide. The initial pH was 3.93 pH and was adjusted to pH 7.74.

A 1% (w/v) Polysorbate 20 solution was prepared by dissolving 0.5 g of Polysorbate 20 in 50 mL of WFI. 2.4 mL of the 1% (w/v) Polysorbate 20 solution was added to the Histidine/Sucrose solution while stirring. 615 mg of BT5528 was weighed out and added slowly to the solution with continuous stirring. Complete dissolution of BT5528 took approximately 120 minutes. After 90 minutes of stirring the API appeared to have adhered to both the bottom and the sides of the beaker. The additional 30 minutes of stirring time resulted in a colourless solution free from visible particulates.

The pH of the solution was measured and adjusted to target (pH 6.8-7.2) with 0.1 M hydrochloric acid. The pH was pH 7.83 after the addition of API and was adjusted to 7.10 pH using 0.1 M HCl solution. The solution was transferred to a 100 mL and a 20 mL volumetric flask, neither fully, and made to final volume with WFI then returned to the original beaker and stirred to mix.

A sample of the solution was collected pre-filtration and the remainder filtered through a single 0.22 μm PES syringe filter, a sample was collected post-filtration for analysis. The filtrate was clear, colourless with no visible particulates.

### 3.2. Lyophilization

The solution was filled into 2 mL vials at a volume of 1 mL and partially stoppered using 13 mm freeze drying stoppers. A single vial located in the center and at the front was probed to monitor product temperature throughout the cycle. The area around the vials was packed out with 2 mL vials containing 1 mL of placebo solution. The vials were lyophilized using the cycle in Table 1-8.

The progress of the cycle was monitored on the basis of shelf and product temperature (thermocouple probes) and chamber pressure measured by Pirani gauge and capacitance manometer.

TABLE 1-8
Lyophilization cycle

| | Cycle | Temperature | Pressure | Time | Ramp rate |
|---|---|---|---|---|---|
| Step | Stage | (° C.) | (mTorr) | (mins) | (° C./min) |
| 1 | Load | 5 | N/A | N/A | N/A |
| 2 | Hold | 5 | N/A | 30 | N/A |
| 3 | Ramp | −40 | N/A | 180 | 0.25 |
| 4 | Freezing | −40 | N/A | 180 | N/A |
| 5 | Extra freezing | −40 | 100 | 30 | N/A |
| 6 | Ramp | −25 | 100 | 75 | 0.20 |
| 7 | Primary drying | −25 | 100 | 3701 | N/A |
| 8 | Ramp | 25 | 20 | 250 | 0.20 |
| 9 | Secondary drying | 25 | 20 | 866 | N/A |

| | | |
|---|---|---|
| 10 | Finish | Vials stoppered to 722,000 mTorr with (pure) nitrogen |

Total cycle duration ~89 hours (~3.7 days)

The lyophilized plugs were white, well-formed with no signs collapse. There was shrinkage from the fill height observed, however, there was no indication this had affected the structure of the lyophilized plug.

The lyophilized product was placed on stability and tested for 1 month (according to Table 1-9) at accelerated storage of 25° C./60% RH. The lyophilized product was assessed by means of appearance, moisture content, and assay and related substances by UPLC. The reconstituted product was assessed on the basis of reconstitution time, solution appearance, pH and sub-visible particulates (T=0 only).

TABLE 1-9
Assessment of dried product

| | T = 0 | T = 1 | T = 2 | T = 4 |
|---|---|---|---|---|
| Test | (wks) | (wks) | (wks) | (wks) |
| UPLC pre-lyo | ✓ | n.a. | n.a. | n.a. |
| Karl Fischer titration | ✓ | — | ✓ | ✓ |
| UPLC | ✓ | ✓ | ✓ | ✓ |
| Reconstitution time¹ | ✓ | — | ✓ | ✓ |
| Reconstituted pH¹ | ✓ | — | ✓ | ✓ |
| Sub-visible particulates | ✓ | — | — | — |

¹Reconstituted with 1 mL WFI

### 3.3. Results

### 3.3.1. Appearance of Lyophilisate

The appearance of the lyophilisate during the stability study is recorded in Table 1-10.

TABLE 1-10
Appearance of lyophilisate, 001/BCL/18

| Time-point/storage | | Appearance |
|---|---|---|
| Initial (T = 0) | | White lyophilised cake with signs |
| | | of slight shrinkage |
| 25° C./60% RH | T = 1 wk | — |
| | T = 2 wk | White lyophilised cake with signs |
| | | of shrinkage |
| | T = 4 wk | White lyophilised cake with signs |
| | | of shrinkage |

TABLE 1-11
Appearance, reconstitution time and pH, 001/BCL/18

| | | Recon. | | |
|---|---|---|---|---|
| Time-point/storage | | time (secs.) | Appearance | pH |
| Initial (T = 0) | | 108¹ | Clear, colourless solution free from visible particulates. | 7.1 |
| 25° C./60% RH | T = 1 wk | — | — | — |
| | T = 2 wk | 529² | Clear, colourless solution free from visible particulates. | 7.1 |
| | T = 4 wk³ | 386 | Clear, colourless solution, | 7.1 |
| | | 409 | speckles noted on bottom of | |
| | | | vial. | |

¹On reconstitution a thick white foam was evident at the bottom of the vial. This should be monitored at future timepoints.
²On reconstitution the vial was swirled for a few minutes before sitting on the bench for the remaining time. Small oily spots were noted on the bottom of the vial, no white solid noted.
³On reconstitution the vial was swirled for a few minutes before sitting on the bench for the remaining time. Speckles of potential product still noted adhering to the bottom of the vial. A further vial was then used to perform a recon experiment. This vial was left for 30 minutes however speckles were still present. Photographs were taken at each stage of swirling and settling during the reconstitution analysis, however it was difficult to show the presence of the speckles due to the small fill volume.

### 3.3.3. Moisture Content

TABLE 1-12
Moisture content, 001/BCL/18

| | |
|---|---|
| Time-point/storage | Moisture (%) |

| Initial (T = 0) | | 1.94¹ |
|---|---|---|
| 25° C./60% RH | T = 1 wk | — |
| | T = 2 wk | 1.44 |
| | T = 4 wk | 2.11¹ |

¹A large variation was noted for moisture content at the T = 0 and T = 4 week timpoints. Clients contacted at T = 0 regarding this. Variation could be due to differences in vial position within the freeze-dryer and differences in manual fill volumes.

### 3.3.4. Sub-Visible Particulates

TABLE 1-13
Sub-visible particulates, 001/BCL/18

| | | Count/vial¹ | | |
|---|---|---|---|---|
| Time-point/storage | | ≥10 μm | ≥25 μm | Pass/Fail² |
| Initial (T = 0) | | 45 | 1 | Pass |
| 25° C./60% RH | T = 1 wk | — | — | — |
| | T = 2 wk | — | — | — |
| | T = 4 wk | — | — | — |

¹Where fill volume is 1 mL
²Number particles with diameter ≥10 μm per vial <6000 = PASS and number particles with diameter ≥25 μm per vial <600 = PASS

### 3.3.5. Pre-Lyo Assay

TABLE 1-14
Pre and Post Filtration Bulk Solution assay, 001/BCL/18

| | Content | Rec./theory¹ | Rec./Pre² |
|---|---|---|---|
| Time-point/storage | (mg/mL) | (%) | (%) |
| Pre Filtration | 3.79 | 94.8 | — |
| Post Filtration | 3.65 | 91.3 | 96.3 |

¹As percentage of theoretical 4 mg/mL.
²As percentage of pre filtration result.

### 3.3.6. Pre-Lyophildzation Related Substances

TABLE 1-15
Pre and post lyophilization related substances

Sample and amount (% area)

| RRT | Pre Filtration | Post Filtratioa |
|---|---|---|
| 0.96 | 0.36 | 0.26 |
| 0.96 | 1.65 | 1.61 |
| 1.07 | 0.58 | 0.51 |
| 1.08 | 0.38 | 0.36 |
| 1.20 | 0.11 | 0.14 |
| 1.27 | 0.16 | 0.11 |
| 1.29 | 0.13 | 0.11 |
| Total¹ | 3.37 | 3.10 |

¹Sum of related substances ≥0.10%

### 3.3.7. Assay Vial Content

TABLE 1-16
Assay, 001/BCL/18

| | | Vial 1 | Vial 2 | Mean content | Rec./theory¹ | Rec./T = 0² |
|---|---|---|---|---|---|---|
| Time-point/storage | | (mg/vial) | (mg/vial) | (mg/vial) | (%) | (%) |
| Initial (T = 0) | | 3.7302 | 3.7607 | 3.75 | 93.6 | — |
| 25° C./60% RH | T = 1 wk | 3.7816 | 3.7132 | 3.75 | 93.7 | 99.9 |
| | T = 2 wk | 3.8873 | 3.7879 | 3.84 | 95.9 | 102.3 |
| | T = 4 wk | 3.8077 | 3.8044 | 3.81 | 95.2 | 101.5 |

¹As percentage of theoretical 5 mg/vial.
²As percentage of initial (T = 0) result.

### 3.3.8. Related Substances

TABLE 1-17
Purity/related substances (area %), 25° C./60% RH

Time-point and amount (% area)

| RRT | Initial (T = 0) | 1 wk | 2 wk | 4 wk |
|---|---|---|---|---|
| 0.96 | 0.31 | 0.10 | <LOQ | 0.28 |
| 0.98 | 1.65 | 1.30 | 1.46 | 1.59 |
| 1.07 | 0.58 | 0.69 | ND | 0.49 |
| 1.08 | 0.39 | 0.53 | 0.66 | 0.75 |
| 1.14 | ND | ND | 0.14 | ND |
| 1.27 | 0.14 | ND | ND | 0.13 |
| 1.28 | 0.12 | 0.21 | 0.21 | 0.16 |
| 1.43 | ND | ND | ND | 0.11 |
| Total¹ | 3.19 | 2.83 | 2.47 | 3.51 |

¹Sum of related substances ≥0.10%

### 3.3.9. Discussion and Conclusions

The cycle parameters for the test lyophilization were found to be suitable for the lead formulation; the lyophilized plugs were white with slight shrinkage. There was no change in the appearance of the lyophilized plug on stability for 4 weeks at 25° C./60% RH. The pH of the reconstituted solution remained at pH 7.1 over the course of the study, however, there was variability in the reconstitution time and oily droplets on the surface of the vials were noted. The reconstitution procedure and appearance of the reconstituted product will be further examined during lyophilization cycle optimisation.

The assay value remained consistent over the 4 week stability study but it should be noted the concentration was 93-95% of theoretical. As the result pre-filtration was also low this suggests adsorption of the API to the glassware during compounding as opposed to a loss on filtration.

The related substances data was comparable at T=0 and T=4 weeks, 3.19% and 3.51% respectively, however, there was a degree of variability in the related substances data from 2.47 to 3.51%.

The lead formulation has been shown to be stable when stored at 25° C./60% RH and therefore was progressed to lyophilization cycle optimisation.

### 4. Stage 6—Lyophilization Cycle Optimisation

The stability data for the lyophilized product generated from the test lyophilization cycle showed the product to be stable during storage at 25° C./60% RH. The first optimisation cycle run used a primary drying shelf temperature of −20° C. with the aim of reducing the duration of primary drying.

A small number of vials were filled (18), to provide sufficient vial numbers for testing of the lyophilized product and a reconstitution stability assessment.

The solution at 4 mg/mL was filled at a volume of 5.3 mL into 10 mL clear Type I glass vials. The lyophilized product was reconstituted with 5.3 mL of WFI to achieve the target concentration of 4 mg/mL.

To assess if the use of a hydrophobic coating prevents adsorption of the product to the vial surface, as observed during test lyophilization stability, 2 mL TopLyo™ vials were also used. A small number of vials, 5, were filled with a 0.5 mL volume and lyophilized in the same cycle.

### 4.1. Lyophilization Optimisation Cycle 1

### 4.1.1. Solution Preparation

A 150 mL bulk solution was prepared as follows:
Approximately 100 mL of WFI was added to a 200 mL beaker containing a magnetic stirrer bar, to this 786.3 mg of histidine HCl was added with rinsing and stirred until dissolved which took 5 minutes.
Once dissolved, 9.0004 g of sucrose was weighed out and added with rinsing to the beaker then stirred magnetically until dissolved. The solution was stirred for 5 minutes until the sucrose had fully dissolved.
The pH of the solution was measured and adjusted to the target pH of pH 7.7-8.1 with 1 M sodium hydroxide before 3 mL of 1% (w/v) Polysorbate 20 was added. The solution was stirred for 5 minutes until homogeneous.
651 mg of BT5528 was weighed out and added slowly with rinsing to the compounding vessel. The solution was stirred magnetically for 90 minutes, small quantities of the API appeared to have adsorbed onto the surface of the beaker. It should be noted the API appears to agglomerate during the dissolution process initially before dissolving fully.
The pH of the solution was measured and adjusted to pH 7.03 with 0.1 M HCl (target pH is pH 6.8-7.2). The solution was then transferred to a 100 mL and a 50 mL volumetric flask, neither fully, and made to volume with WFI. The solution was returned to the original beaker and stirred to mix.
The solution was filtered through a single 0.22 μm PES syringe filter with pre- and postfiltration samples collected for analysis. The resultant filtrate was a clear, colourless solution free from visible particulates.

### 4.1.2. Lyophilization

The solution was filled into 10 mL Type I clear glass vials at a volume of 5.3 mL and also into both standard and TopLyo™ (silanised vials) at a volume of 0.5 mL to determine if the use of a hydrophobic surface reduced adsorption of BT5528 onto the surface.

A single vial located in the center of the tray was probed to monitor produce temperature throughout the cycle. The progress of the cycle was monitored on the basis of temperature (shelf/product probe) and chamber pressure (Capacitance manometer/Pirani gauge) to determine the end point of primary and secondary drying.

The vials were lyophilized directly from the shelf using the cycle in Table 1-18.

TABLE 1-18
Lyophilization cycle

| | Cycle | Temperature | Pressure | Time | Ramp rate |
|---|---|---|---|---|---|
| Step | Stage | (° C.) | (mTorr) | (mins) | (° C./min) |
| 1 | Load | 5 | N/A | N/A | N/A |
| 2 | Hold | 5 | N/A | 30 | N/A |
| 3 | Ramp | −40 | N/A | 180 | 0.25 |
| 4 | Freezing | −40 | N/A | 180 | N/A |
| 5 | Extra freezing | −40 | 100 | 30 | N/A |
| 6 | Ramp | −20 | 100 | 100 | 0.20 |
| 7 | Primary drying | −20 | 100 | 4658 | N/A |
| 8 | Ramp | 25 | 20 | 225 | 0.20 |
| 9 | Secondary drying | 25 | 20 | 368 | N/A |

| | | |
|---|---|---|
| 10 | Finish | Vials stoppered to 722,000 mTorr with (pure) nitrogen |

Total cycle duration ~96.2 hears (~4 days)

The lyophilized plugs were white, well-formed and with slight shrinkage. The shrinkage appears to be an inherent feature of the formulation as this was also observed in the test lyophilization cycle with a primary drying temperature of −25° C. The lyophilized plugs in the TopLyo™ vials showed gross collapse and it was therefore not possible to test the product. The collapse is likely due to the extended drying time of the 5.3 mL fill volume in the 10 mL vial compared to the 0.5 mL fill in a 2 mL vial leading to the product temperature increasing above the collapse temperature.

### 4.1.3. Results

The batch details are shown in Table 1-19. Batch 002/BCL/18 was lyophilized in optimisation cycle 1.

TABLE 1-19
Drug product composition (Batch number and Formulation details)

| | |
|---|---|
| 002/BCL/18 | 4 mg/mL BT5528, 5.24 mg/mL histidine HCl, |
| | 60 mg/mL sucrose, 0.2 mg/mL polysorbate |
| | 20 (5.3 mL fill - 21.2 mg/vial) |

### 4.1.3.1. Appearance of Solution, pH and Reconstitution Time

TABLE 1-20
Appearance of solution, pH reconstitution time

| | | | Reconstitution |
|---|---|---|---|
| Sample | Appearance | pH | time (minutes) |
| 002/BCL/18 | A clear colourless solution | 7.1 | 12.03¹ |
| | free from visible particles | | |

¹In order to reconstitute vigorous shaking was required. After the time recorded no large clumps remained however oily deposits of material could still be seen suggesting the product is adhering to vial, FIG. 1. Vortex mixing was required to obtain foil dissolution.

TABLE 1-21
Appearance lyophilized plug

| Sample | Appearance |
|---|---|
| 002/BCL/18 | A homogenous white lyophilised cake |
| | with evident shrinkage |

### 4.1.3.2. Sub-Visible Particulates

TABLE 1-22
Sub-visible particulates

| | 10 μm | 25 μm | |
|---|---|---|---|
| Sample | (counts/vial)¹ | (counts/vial)¹ | Pass/Fail¹ |
| 002/BCL/18 | 173 | 4 | Pass |

¹Where fill volume is 5.3 ml.
²Number particles with diameter ≥10 μm per vial ≤6000 = PASS and number particles with diameter ≥25 μm per vial ≤600 = PASS

### 4.1.3.3. Moisture Content

TABLE 1-23
Water content

| | Mean water |
|---|---|
| Sample | (% w/w) |
| 002/BCL/18 | 2.50 |

### 4.1.3.4. Assay (Vial Content)

TABLE 1-24
Assay

| | | Pre | Post | | | | |
|---|---|---|---|---|---|---|---|
| | | filtration | Filtration | Post | | | |
| | | Assay | Assay | Rec./theory¹ | Assay | Rec./theory¹ | Rec./Post² |
| Sample | | (mg/mL) | (mg/mL) | (%) | (mg/mL) | (%) | (%) |
| 002/BCL/18 | Vial 1 | 4.2092 | 4.0521 | 96.27 | 4.2369 | 105.92 | 104.56 |
| | Vial 2 | | | | 4.1854 | 104.63 | 103.29 |

¹As percentage of theoretical 4 mg/mL
²As percentage of post filtration result.

### 4.1.3.5. Purity/Related Substances

TABLE 1-25
Purity/related substances

| | Amount (% area/area) | | |
|---|---|---|---|
| RRT | Pre filtration | Post filtration | 002/BCL/18 |
| 0.97 | 0.92 | 0.90 | 0.86 |
| 0.98 | 1.78 | 1.40 | 1.75 |
| BT5528 | 96.07 | 96.40 | 96.17 |
| 1.09 | 0.76 | 0.85 | 0.78 |
| 1.16 | 0.13 | 0.14 | 0.11 |
| 1.27 | 1.11 | 0.10 | 0.12 |
| 1.30 | 0.16 | 0.15 | 0.15 |
| Total¹ | 3.86 | 3.54 | 3.77 |

¹Sum of related substances ≥0.10%

### 4.1.4. Discussion and Conclusions

The appearance of the lyophilized plugs showed a primary drying temperature of −20° C. to be suitable for the lead formulation and maintained the product temperature below the collapse temperature during the initial stages of primary drying. The temperature exceeded the collapse temperature towards the end of primary drying as the drying neared completion. It would be recommended that for the second optimisation cycle the primary drying temperature is further increased to −15° C. as there was no sign of product collapse.

Reconstitution of the lyophilized product was similar to the test lyophilization product with oily droplets forming and vortex mixing required for reconstitution. The use of the vortex caused excessive foaming of the product due to the presence of Polysorbate 20 in the formulation. The reconstitution time was approximately 12 minutes which was above the target time of 10 minutes.

The assay value was at target indicating there was no loss of API due to adsorption to glassware during compounding or during filtration. The purity/related substances data was similar post-lyophilization as for pre- and post-filtration.

### 4.2. Lyophilization Optimisation Cycle 2

The first optimisation cycle run used a primary drying shelf temperature of −20° C. which reduced the duration of primary drying. The lyophilized product was white, well-formed with no signs of collapse. It was therefore recommended for the second lyophilization cycle the shelf temperature was increased to −15° C. to further reduce the duration of primary drying.

A small number of vials were filled, 18, to provide sufficient vial numbers for testing of the lyophilized product.

The solution at 4 mg/mL was filled at a volume of 5.3 mL into 10 mL clear Type I glass vials. The lyophilized product was reconstituted with 5.3 mL of WFI to achieve the target concentration of 4 mg/mL.

To assess if the use of a hydrophobic coating prevented adsorption of the product to the vial surface, as observed during test lyophilization stability, 2 mL TopLyo™ vials were used. A small number of vials, 5, were filled with a 0.5 mL volume of the solution and lyophilized in the same cycle. A small number of 2 mL vials with no coating were also filled as a comparison to the TopLyo™ vials, as collapse was observed during cycle optimisation 1 and an assessment of the impact of a silanised coating was therefore not possible.

### 4.2.1. Solution Preparation

A 120 mL bulk solution was prepared as follows.

Approximately 80 mL of WFI was added to a 200 mL beaker containing a magnetic stirrer bar, to this 628.8 mg of histidine HCl was added with rinsing and stirred until dissolved which took 5 minutes.

Once dissolved, 7.2 g of sucrose was weighed out and added with rinsing to the beaker then stirred magnetically until dissolved. The solution was stirred for 5 minutes until the sucrose had fully dissolved.

The pH of the solution was measured and adjusted to the target pH of pH 7.7-8.1 with 1 M sodium hydroxide before 2.4 mL of 1% (w/v) Polysorbate 20 was added. The solution was stirred for 5 minutes until homogeneous.

615 mg of BT5528 was weighed out and added slowly with rinsing to the compounding vessel. The solution was stirred magnetically for 120 minutes, after 90 minutes the API appeared to have adsorbed onto the surface of the bottom of the beaker and also the magnetic stirrer bar. The additional stirring time resulted in a colourless solution free from particulates.

The pH of the solution was measured and adjusted to pH 7.1 (target pH is pH 6.8-7.2). The solution was then transferred to a 100 mL and a 20 mL volumetric flask, neither fully, and made to volume with WFI. The solution was returned to the original beaker and stirred to mix.

The solution was filtered through a single 0.22 μm PES syringe filter with pre- and postfiltration samples collected for analysis. The resultant filtrate was a clear, colourless solution free from visible particulates.

### 4.2.2. Lyophilization

A single vial located in the center of the tray was probed to monitor produce temperature throughout the cycle. The progress of the cycle was monitored on the basis of temperature (shelf/product probe) and pressure (Capacitance manometer/Pirani gauge) differentials to determine the end point of primary and secondary drying.

The vials were lyophilized directly from the shelf using the cycle in Table 1-26. Due to a product probe fault, temperature data was not obtained during the lyophilization cycle. Convergence of the Pirani gauge and capacitance manometer was used to indicate completion of primary and secondary drying.

TABLE 1-26
Lyophilization cycle

| | | Temperature | Pressure | Time | Ramp rate |
|---|---|---|---|---|---|
| Step | Cycle Stage | (° C.) | (mTorr) | (mins) | (° C./min) |
| 1 | Load | 5 | N/A | N/A | N/A |
| 2 | Hold | 5 | N/A | 30 | N/A |
| 3 | Ramp | −40 | N/A | 180 | 0.25 |
| 4 | Freezing | −40 | N/A | 180 | N/A |
| 5 | Extra freezing¹ | −40 | 100 | 30 | N/A |
| 6 | Ramp | −15 | 100 | 125 | 0.20 |
| 7 | Primary drying | −15 | 100 | 300 | N/A |
| 8 | Ramp | 25 | 20 | 250 | 0.20 |
| 9 | Secondary | 25 | 20 | 866 | N/A |
| | drying | | | | |

| | | |
|---|---|---|
| 10 | Finish | Vials stoppered to 722,000 mTorr |
| | | with (pure) nitrogen |

Total cycle duration ~89 hours (~3.7 days)

### 4.2.3. Results

The batch details are shown in Table 1-28.

TABLE 1-27
Drug product composition (Batch number and Formulation details)

| | |
|---|---|
| 003/BCL/18 | 4 mg/mL BT5528, 5.24 mg/mL histidine HCl, |
| | 60 mg/mL sucrose, 0.2 mg/mL polysorbate 20 |
| | (5.3 mL fill - 21.2 mg/vial) |

4.2.3.1. Appearance of Solution, pH and Reconstitution Time

TABLE 1-28
Appearance of solution, pH reconstitution time

| | | | Reconstitution time |
|---|---|---|---|
| Sample | Appearance | pH | (minutes) |
| 003/BCL/18 | A clear colourless solution | 6.7 | 11.14 |
| | free from visible particles | | |

¹In order to reconstitute vigorous shaking was required. After the time recorded no large clumps remained however oily deposits of material could still be seen suggesting the product is adhering to vial. Vortex mixing was required to obtain full dissolution.

TABLE 1-29
Appearance lyophilized plug

| Sample | Appearance |
|---|---|
| 003/BCL/18 | A homogenous white lyophilised cake |
| | with evident shrinkage |

### 4.2.3.2. Sub-Visible Particulates

TABLE 1-30
Sub-visible particulates

| | 10 μm | 25 μm | |
|---|---|---|---|
| Sample | (counts/vial)¹ | (counts/vial)¹ | Pass/Fail² |
| 003/BCL/18 | 209 | 4 | Pass |

¹Where fill volume is 5.3 mL
²Number particles with diameter ≥10 μm per vial ≤6000 = PASS and number particles with diameter ≥25 μm per vial ≤600 = PASS

### 4.2.3.3. Moisture Content

TABLE 1-31
Water content

| | Mean water |
|---|---|
| Sample | (% w/w) |
| 003/BCL/18 | 2.13 |

### 4.2.3.4. Assay (Vial Content)

TABLE 1-32
Assay

| | | Pre | Post | | | | |
|---|---|---|---|---|---|---|---|
| | | filtration | Filtration | Post | | | |
| | | Assay | Assay | Rec./theory¹ | Assay | Rec./theory¹ | Rec./Post² |
| Sample | | (mg/mL) | (mg/mL) | (%) | (mg/mL) | (%) | (%) |
| 003/BCL/18 | Vial 1 | 3.9008 | 3.9849 | 98.57 | 102.16 | 21.4138 | 4.0403 |
| | Vial 2 | | | | | 22.6418 | 4.2720 |

¹As percentage of theoretical 4 mg/mL
²As percentage of post filtration result.

### 4.2.3.5. Purity/Related Substances

TABLE 1-33
Purity/related substances Batch 003/BCL/18

| | Amount (% area/area) | | |
|---|---|---|---|
| RRT | Pre filtration | Post filtration | 003/BCL/18 |
| 0.96 | 0.19 | 0.19 | 0.18 |
| BT5528 | 98.89 | 98.85 | 98.83 |
| 1.12 | 0.72 | 0.81 | 0.77 |
| Total¹ | 0.91 | 1.00 | 1.05 |

¹Sum of related substances ≥0.10%

### 3.2.4. Discussion and Conclusions

The appearance of the lyophilized plugs showed a primary drying temperature of −15° C. to be suitable for the lead formulation and maintained the product temperature below the collapse temperature during the initial stages of primary drying. The temperature exceeded the collapse temperature towards the end of primary drying as the drying neared completion.

Reconstitution of the lyophilized product was similar to the test lyophilization product with oily droplets forming and vortex mixing required for reconstitution. The use of the vortex caused excessive foaming of the product due to the presence of Polysorbate 20 in the formulation. The reconstitution time was approximately 11 minutes which is above the target time of 10 minutes.

Reconstitution of the lyophilized plug in the TopLyo™ vials was longer than the standard Type I clear glass 2 mL vials and greater adsorption of the API to the surface was observed. This showed that the use of silanised vials would not improve the reconstitution or assay recovery values for the lyophilized product.

The assay value was at target indicating there was no loss of API due to adsorption to glassware during compounding or during filtration. The purity/related substances data was similar post-lyophilization as for pre- and post-filtration, however, it should be noted the total related substances was significantly less than optimisation cycle 1.

The moisture content value was similar for this cycle, 2.13% (w/w), when compared to lyophilization optimisation cycle 1 (2.50% (w/w)).

An assessment of reconstitution of the lyophilized product in the 10 mL vials (21.2 mg/vial BT5528) was carried out using an acidic solution. As BT5528 is a basic protein, adjusting the pH is likely to improve reconstitution. Reconstitution with a dilute acid rather than WFI to achieve a lower buffer pH was hypothesized to increase the rate of solubilization after reconstitution. Improved reconstitution with an acidic solution would indicate a change in the target pH of the formulation pre-lyophilization to a lower value (e.g. pH 6 or 6.5 as compared to the current pH 7.1) would improve reconstitution of the lyophilized product with WFI alone.

Single vials of lyophilized BT5528 product were reconstituted with either 5.3 mL of 0.01 M HCl or 0.02 M HCl and the appearance noted over a period of 15 minutes. The reconstitution time and appearance of the reconstituted product, with respect to oily droplet formation, was improved with the 0.02 M HCl solution. This suggested a more acidic pH in the lyophilized product may improve the reconstitution characteristics.

A second hypothesis for the reconstitution challenges encountered was a high salt concentration in the formulation as a result of the compounding process. Sodium hydroxide was used to adjust the pH from ˜pH 3.5 to pH 7.7-8.1 before API dissolution followed by the use of hydrochloric acid for pH adjustment to target pH 6.8-7.2.

### 5. Histidine/pH Assessment

The use of an acidic solution for reconstitution improved the reconstitution time and appearance. To investigate if a lower pH pre-lyophilization improved the reconstitution time and assay result, three solutions were prepared at pH 6, pH 6.5 and pH 7.0, lyophilized using the cycle in Table 36 then assessed post-lyophilization.

Additionally, the compounding process was amended to reduce the use of sodium hydroxide for pH adjustment and the sodium chloride content in the final product. The pH of the solutions prepared before addition of BT5528 was not adjusted, and were only adjusted after dissolution of the API.

The formulations are shown in Table 1-34.

TABLE 1-34
Histidine/pH formulation compositions
(Batch number and Formulation details)

| | |
|---|---|
| Formulation 1 | 4 mg/mL BT5528, 5.24 mg/mL histidine HCl, |
| | 60 mg/mL sucrose, 0.2 mg/mL |
| | polysorbate 20, pH 6 |
| Formulation 2 | 2 mg/mL BT5528, 5.24 mg/mL histidine HCl, |
| | 60 mg/mL sucrose, 0.2 mg/mL |
| | polysorbate 20, pH 6.5 |
| Formulation 3 | 2 mg/mL BT5528, 5.24 mg/mL histidine, |
| | 60 mg/mL sucrose, 0.1 mg/mL |
| | polysorbate 20, pH 7 |

### 5.1. Solution Preparation

The solution preparation details for each of the three formulations prepared were as follows.

### Formulation 1—Histidine HCl—pH 6

Approximately 35 mL of WFI was added to a beaker containing a magnetic stirrer bar. To this, 262.1 mg of histidine HCl was added with rinsing and stirred magnetically until dissolved. Once dissolved, 3.0322 g of sucrose was weighed out and added with rinsing then stirred until dissolved. 1 mL of a 1% (w/v) Polysorbate 20 solution was pipetted into the histidine/sucrose solution and stirred until complete dissolution was achieved. The pH of the solution was measured, pH 4.01.

258 mg of BT5528 was weighed out and added slowly then stirred for 90 minutes until fully dissolved. The pH of the solution was measure and adjusted to pH 6.02 with 1 M sodium hydroxide then transferred to a 50 mL volumetric flask and made to volume with WFI. The solution was returned to the original beaker and stirred to mix.

The solution was filtered through a single 0.22 μm PES syringe filter, the filtrate was a clear, colourless solution free from visible particulates.

### Formulation 2—Histidine HCl—pH 6.5

Approximately 35 mL of WFI was added to a beaker containing a magnetic stirrer bar. To this, 262.2 mg of histidine HCl was added with rinsing and stirred magnetically until dissolved. Once dissolved, 3.0360 g of sucrose was weighed out and added with rinsing then stirred until dissolved. 1 mL of a 1% (w/v) Polysorbate 20 solution was pipetted into the histidine/sucrose solution and stirred until complete dissolution was achieved. The pH of the solution was measured, pH 4.01.

257 mg of BT5528 was weighed out and added slowly then stirred for 90 minutes until fully dissolved. The pH of the solution was measure and adjusted to pH 6.49 with 1 M sodium hydroxide then transferred to a 50 mL volumetric flask and made to volume with WFI. The solution was returned to the original beaker and stirred to mix. The solution was filtered through a single 0.22 μm PES syringe filter, the filtrate was a clear, colourless solution free from visible particulates.

### Formulation 3—Histidine—pH 7

Approximately 35 mL of WFI was added to a beaker containing a magnetic stirrer bar. To this, 262.0 mg of histidine was added with rinsing and stirred magnetically until dissolved. Once dissolved, 3.0439 g of sucrose was weighed out and added with rinsing then stirred until dissolved. 1 mL of a 1% (w/v) Polysorbate 20 solution was pipetted into the histidine/sucrose solution and stirred until complete dissolution was achieved. The pH of the solution was measured, pH 7.73.

257 mg of BT5528 was weighed out and added slowly then stirred for 90 minutes until fully dissolved. The pH of the solution was measure and adjusted to pH 6.97 with 1 M hydrochloric acid then transferred to a 50 mL volumetric flask and made to volume with WFI. The solution was returned to the original beaker and stirred to mix.

The solution was filtered through a single 0.22 μm PES syringe filter, the filtrate was a clear, colourless solution free from visible particulates.

### 5.2. Lyophilization

Each of the three formulations prepared were filled into 10 mL Type I clear glass vials at a volume of 5.3 mL.

The vials were loaded into the center of a freeze drying tray and lyophilized directly from the shelf using the cycle in Table 1-35. The progress of the cycle was monitored on the basis of temperature (shelf/product probe) and pressure (Capacitance manometer/Pirani gauge) differentials to determine the end point of primary and secondary drying.

TABLE 1-35
Lyophilization cycle

| | | Temperature | Pressure | Time | Ramp rate |
|---|---|---|---|---|---|
| Step | Cycle Stage | (° C.) | (mTorr) | (mins) | (° C./min) |
| 1 | Load | 5 | N/A | N/A | N/A |
| 2 | Hold | 5 | N/A | 30 | N/A |
| 3 | Ramp | −40 | N/A | 180 | 0.25 |
| 4 | Freezing | −40 | N/A | 180 | N/A |
| 5 | Extra freezing¹ | −40 | 100 | 30 | N/A |
| 6 | Ramp | −15 | 100 | 125 | 0.20 |
| 7 | Primary drying | −15 | 100 | 3739 | N/A |
| 8 | Ramp | 25 | 20 | 200 | 0.20 |
| 9 | Secondary | 25 | 20 | 904 | N/A |
| | drying | | | | |

| | | |
|---|---|---|
| 10 | Finish | Vials stoppered to 722,000 mTorr |
| | | with (pure) nitrogen |

Total cycle duration ~90 hours (~3.7 days)

### 5.3. Results

### 5.3.1. Appearance of Solution, pH and Reconstitution Time

In order to reconstitute, vigorous shaking was required. After the time recorded, no large clumps remained, however sticking of material to vial walls could still be seen suggesting the product was adhering to vial. Vortex mixing was required to obtain complete dissolution.

TABLE 1-36
Appearance of solution, pH reconstitution time

| | | | Reconstitution |
|---|---|---|---|
| | | | time |
| Sample | Appearance | pH | (minutes) |
| Histidine HCl pH 6.0 | A clear colourless solution | 6.0 | 3 mins 04 secs |
| | free from visible particles | | |
| Histidine HCl pH 6.5 | A clear colourless solution | 6.5 | 6 mins 04 secs |
| | free from visible particles | | |
| Histidine pH 7.0 | A clear colourless solution | 7.0 | 8 mins 38 secs |
| | free from visible particles | | |

TABLE 1-37
Appearance of lyophilized plug

| Sample | Appearance |
|---|---|
| Histidine HCl pH 6.0 | White lyophilised cake with evident shrinkage |
| Histidine HCl pH 6.5 | White lyophilised cake with evident shrinkage |
| Histidine pH 7.0 | White lyophilised cake with evident shrinkage |

### 5.3.2. Assay (Vial Content)

TABLE 1-38
Assay

| | | Pre | Post | | | | | |
|---|---|---|---|---|---|---|---|---|
| | | filtration | Filtration | Post | Post | | | |
| | | Assay | Assay | Rec./theory¹ | Rec./Pre | Assay | Rec./theory¹ | Rec./Post² |
| Sample | | (mg/mL) | (mg/mL) | (%) | (%) | (mg/vial) | (%) | (%) |
| pH 6.0 | Vial 1 | 4.3353 | 4.3047 | 107.62 | 99.29 | 3.8921 | 97.30 | 90.42 |
| | Vial 2 | | | | | 3.9510 | 98.78 | 91.78 |
| pH 6.5 | Vial 1 | 4.2583 | 4.2805 | 107.01 | 100.52 | 3.8813 | 97.03 | 90.67 |
| | Vial 2 | | | | | 3.9095 | 97.74 | 91.33 |
| pH 7.0 | Vial 1 | 4.0259 | 4.0095 | 100.24 | 99.59 | 3.5649 | 89.12 | 88.91 |
| | Vial 2 | | | | | 3.6056 | 90.14 | 89.92 |

¹As percentage of theoretical 4 mg/mL
²As percentage of post filtration result.

### 5.3.3. Purity/Related Substances

TABLE 1-39
Purity/related substances Histidine HC1 pH 6.0

| | Amount (% area/area) | | |
|---|---|---|---|
| | | | Histidine HCl |
| RRT | Pre filtration | Post filtration | pH 6.0 |
| 0.96 | 0.29 | 0.25 | 0.33 |
| BT5528 | 98.50 | 98.57 | 98.47 |
| 1.12 | 0.75 | 0.80 | 0.69 |
| 1.16 | 0.12 | <LOQ | 0.12 |
| 1.33 | 0.11 | 0.13 | 0.10 |
| Total¹ | 1.27 | 1.18 | 1.24 |

¹Sum of related substances ≥0.10%

TABLE 1-40
Purity/related substances. Histidine HCl pH 6.5

| | Amount (% area/area) | | |
|---|---|---|---|
| | | | Histidine HCl |
| RRT | Pre filtration | Post filtration | pH 6.5 |
| 0.96 | 0.22 | 0.17 | 0.28 |
| BT5528 | 99.66 | 98.80 | 98.24 |
| 1.10 | ND | 0.44 | 0.48 |
| 1.12 | 0.76 | 0.44 | 0.58 |
| 1.29 | 0.10 | <LOQ | 0.12 |
| 1.33 | <LOQ | <LOQ | 0.10 |
| Total¹ | 1.08 | 1.05 | 1.56 |

¹Sum of related substances ≥0.10%

TABLE 1-41
Purity/related substances. Histidine HCl pH 7.0

| | Amount (% area/area) | | |
|---|---|---|---|
| | | | Histidine HCl |
| RRT | Pre filtration | Post filtration | pH 7.0 |
| 0.94 | | ND | 0.11 |
| 0.96 | 0.30 | 0.29 | 0.34 |
| BT5528 | 98.37 | 98.41 | 98.62 |
| 1.10 | 0.45 | 0.52 | ND |
| 1.12 | 0.65 | 0.56 | 0.69 |
| 1.33 | <LOQ | 0.15 | 0.12 |
| Total¹ | 1.40 | 1.52 | 1.26 |

¹Sum of related substances ≥0.10%

### 5.3.4. Discussion and Conclusions

The appearance of the lyophilized plugs for the three formulations were similar, indicating no effect of pH or compounding on product appearance. The appearance of the lyophilized plugs were the same as for the test lyophilization and optimisation cycles 1 and 2.

The reconstitution time of the plugs increased with pH, from 3 minutes 4 seconds for pH 6 to 8 minutes 38 seconds for pH 7.0. This showed the pH of the reconstituted solution had an impact on the reconstitution time. Additionally, the amended compounding process (reducing the use of sodium hydroxide) appeared to have improved the reconstitution time with times of <9 minutes for the three formulations compared to >11 minutes previously. However, vortex mixing was still required to achieve reconstitution and oily droplets were visible on the vial surface. Therefore pH and the presence of salt are not the only factors involved in adsorption of the API to the surface of the vials.

The purity/related substances for the three formulations were similar (Table 1-39 to Table 1-41). The assay data showed no adsorptive loss of BT5528 during filtration (Table 1-38). A higher assay (vial content) was observed at pH 6 compared to pH 7 indicating a lower pH pre-lyophilization improved BT5528 recovery.

Oily droplets were still visible despite the shorter reconstitution time and improved recovery at pH 6 compared to pH 7 and as a result further work was carried out assessing alternative sugars and a lower secondary drying temperature to increase the moisture content of the final product. BT5528 is reported to have good solubility in water and it was hypothesized that the long reconstitution time, and oily droplet formation, may be due to over-drying of the peptide during the lyophilization process. This may have resulted in the loss of the hydration shell and changed the peptide conformation in the lyophilized product.

### 6. Sugar Formulation Screen

A change in pH of the formulation pre-lyophilization did not prevent the formation of oily droplets on the surface of the vials during reconstitution but did improve the reconstitution time (pH 6 was shorter than pH 7).

Due to the adsorptive nature of the formulations lyophilized during optimisation cycle 3, 5 formulations with a range of sugars and an alternative surfactant would be assessed. The compositions of the formulations are shown in Table 1-42.

As discussed in section 5.3.4 it was hypothesized the product may have been over-dried resulting in removal of the peptide hydration shell, therefore a lower (0° C.) secondary drying time was also investigated in addition to the sugar/surfactant screen.

TABLE 1-42
Drug product composition (Batch number and Formulation details)

| | |
|---|---|
| Formulation 1 | 4 mg/mL BT5538, 5.24 mg/mL histidine HCl, |
| | 60 mg/mL sucrose, 0.2 mg/mL |
| | polysorbate 20 (5.3 mL fill - 21.2 mg/vial) 20 mL vial |
| Formulation 2 | 4 mg/mL BT5528, 5.24 mg/mL histidine HCl, |
| | 60 mg/mL sucrose, 0.2 mg/mL |
| | polysorbate 80 (5.3 mL fill - 21.2 mg/vial) 10 mL vial |
| Formulation 3 | 4 mg/mL BT5528, 5.24 mg/mL histidine HCl, |
| | 60 mg/mL trehalose, 0.2 mg/mL |
| | polysorbate 20 (5.3 mL fill - 21.2 mg/vial) 10 mL vial |
| Formulation 4 | 4 mg/mL BT5528, 5.24 mg/mL histidine HCl, |
| | 60 mg/mL dextrose, 0.2 mg/mL |
| | polysorbate 20 (5.3 mL fill - 21.2. mg/vial) 10 mL vial |
| Formulation 5 | 2 mg/mL BT5528, 5.24 mg/mL histidine HCl, |
| | 60 mg/mL sucrose, 0.2 mg/mL |
| | polysorbate 20 (10.6 mL fill - 21.2 mg/vial) 20 mL vial |

### 6.1. Solution Preparation

Each of the formulations was prepared in the same manner with the exception of Formulation 5 which is detailed separately. Approximately 35 mL of WFI was added to a beaker containing a magnetic stirrer bar. To this, ˜262 mg of histidine was added with rinsing and stirred magnetically until dissolved. Once dissolved, ˜3 g of sucrose/trehalose/dextrose (Formulations 1-2, 3 and 4 respectively) was weighed out and added with rinsing then stirred until dissolved. 1 mL of a 1% (w/v) Polysorbate 20 solution was pipetted into the histidine/sucrose solution for formulations 1, 3 and 4 and 1 mL of a 1% (w/v) Polysorbate 80 solution for formulation 2. The solutions were stirred until complete dissolution was achieved. The pH of each solution was measured.

Formulation 5 was prepared as follows: approximately 70 mL of WFI was added to a beaker containing a magnetic stirrer bar. To this, 524.9 mg of histidine was added with rinsing and stirred magnetically until dissolved. Once dissolved, 6.0031 g of sucrose was weighed out and added with rinsing then stirred until dissolved. 2 mL of a 1% (w/v) Polysorbate 20 solution was pipetted into the histidine/sucrose solution and stirred until complete dissolution was achieved. The pH of the solution was measured.

To each of the five formulations, ˜257 mg of BT5528 was weighed out and added slowly then stirred for 90 minutes until fully dissolved. It should be noted that during addition/dissolution the BT5528 adhered to the sides of the beakers and were gel-like in appearance. The BT5528 was removed from the sides of the beaker using a pipette resulting in the API dissolving fully in the solution. The pH of the solutions was measure and adjusted to pH 6.5 with 1 M sodium hydroxide then transferred to a 50 mL (Formulations 1-4) or 100 mL (Formulation 5) volumetric flask and made to volume with WFI. The solutions were returned to the original beakers and stirred to mix.

Each the solution was filtered through a single 0.22 μm PES syringe filter, the filtrate was a clear, colourless solution free from visible particulates.

TABLE 1-43
Solution pH (Batch number, Buffer pH, and Final pH)

| | | |
|---|---|---|
| Formulation 1 | 7.74 | 6.51 |
| Formulation 2 | 7.75 | 6.52 |
| Formulation 3 | 7.65 | 6.52 |
| Formulation 4 | 7.76 | 6.51 |
| Formulation 5 | 7.75 | 6.52 |

### 6.2. Lyophilzation

The volume of solution filled and vial type are shown in Table 1-44.

TABLE 1-44
Vial type and fill volume (Batch number,
Vial (mL), and Fill volume (mL))

| | | |
|---|---|---|
| Formulation 1 | 20 | 5.3 |
| Formulation 2 | 10 | 5.3 |
| Formulation 3 | 10 | 5.3 |
| Formulation 4 | 10 | 5.3 |
| Formulation 5 | 20 | 10.6 |

TABLE 1-45
Lyophilization cycle parameters

| | Cycle | Temperature | Pressure | Time | Ramp rate |
|---|---|---|---|---|---|
| Step | Stage | (° C.) | (mTorr) | (mins) | (° C./min) |
| 1 | Load | 5 | N/A | N/A | N/A |
| 2 | Hold | 5 | N/A | 30 | N/A |
| 3 | Ramp | −40 | N/A | 180 | 0.25 |
| 4 | Freezing | −40 | N/A | 180 | N/A |
| 5 | Extra Freezing | −40 | 100 | 30 | N/A |
| 6 | Ramp | −25 | 100 | 75 | 0.20 |
| 7 | Primary drying | −25 | 100 | 6450 | N/A |
| 8 | Ramp | 0 | 20 | 125 | 0.20 |
| 9 | Secondary drying | 0 | 20 | 1005 | N/A |

| | | |
|---|---|---|
| 10 | Finish | Vials stoppered to 722,000 mTorr with (pure) nitrogen |

Total cycle duration ~135 hrs (~5.6 days)

The lyophilized plugs from Formulation 1 were white, well-formed and homogenous with no signs of shrinkage or collapse. The lyophilized plugs for Formulations 2, 4 and 5 were white, homogeneous but with signs of shrinkage.

Formulation 3 (trehalose) showed gross collapse and melt back of the plug.

The thermal characteristics of the trehalose and dextrose formulations were not determined before the solutions were lyophilized, however, conservative cycle parameters were used. The collapse temperature of trehalose reported in the literature is approximately −28° C., which is higher than reported for both sucrose (−31° C.) and dextrose (−41° C.) and therefore a primary drying shelf temperature of −25° C. was expected to be suitable. The product probe data showed the temperature of the product to be approximately −38° C. during the initial stages of primary drying and would therefore not be expected to collapse.

Formulation 4, containing dextrose, did not collapse and thus it was not possible to determine the reason for the collapse observed.

### 6.3. Results

### 6.3.1. Appearance of Solution, pH and Reconstitution Time

TABLE 1-46
Appearance of solution, pH reconstitution time

| | | | Reconstitution |
|---|---|---|---|
| Sample | Appearance¹ | pH | time (minutes) |
| Formulation 1 | A clear colourless solution, | 6.4 | 1 mins 35 secs |
| 4 mg/mL | adsorption noted on side walls. | | |
| Formulation 1 | A clear colourless solution, | 6.5 | 3 mins 17 secs |
| 3 mg/mL | adsorption noted on side walls. | | |
| Formulation 1 | A clear colourless solution, | 6.5 | 7 mins 27 secs |
| 1.3 mg/mL | adsorption noted on side walls. | | |
| Formulation 2 | A clear colourless solution, | 6.5 | 5 mins 40 secs |
| | adsorption noted on side walls. | | |
| Formulation 3 | A clear colourless solution, | 6.5 | 16 mins 00 secs |
| | cake did not dissolve. | | |
| Formulation 4 | A clear colourless solution, | 6.5 | 6 mins 40 secs |
| | adsorption noted on side walls. | | |
| Formulation 5 | A clear colourless solution, | 6.5 | 4 mins 03 secs |
| | less adsorption on side walls. | | |

During the optimisation cycle analysis, adsorption was noted for all formulations. Formulations 1 to 4 had significant adsorptive properties with the drug product adsorbing onto the glass vial above and in the reconstituted solution. Formulation 5 (2 mg/mL BT5528) produced the best results with drug product adsorbing only slightly above the solution and no adsorption noted within the solution. Variability in reconstitution time derives from the analytical procedure deviating throughout the testing of formulation 1. This occurred due to the severe adsorptive nature of formulation 1. For the 4 mg/mL solution agitation occurred immediately after addition of reconstitution medium producing a severe adsorptive spread above the solution. A settle period before agitation was performed for the 2 mg/mL producing a better reconstitution performance although adsorption was still noted. For the 1.3 mg/mL solution a settle period of 5 minutes was performed to minimize the agitation required. Adsorption did not improve with this inflated settle period. For formulation 2 onwards a settle period of 2 minutes, 1 minute agitation and a final 1 minute settle period before agitation performed until dissolution. Formulation 5 performed the best out of the formulations and for cycle 4 optimisation a typical reconstitution method will be performed for this formulation (agitation immediately after addition of reconstitution medium).

TABLE 1-47
Appearance lyophilized plug

| Sample | Appearance¹ |
|---|---|
| Formulation 1 | A homogenous white lyophilised cake |
| Formulation 2 | A homogenous white lyophilised cake |
| | with evident shrinkage |
| Formulation 3 | A white disc at the centre of the |
| | vial with evident gross collapse |
| Formulation 4 | A homogenous white lyophilised cake |
| | with evident shrinkage |
| Formulation 5 | A homogenous white lyophilised cake |
| | with evident shrinkage |

¹Refer to appendices for photographs lyophilised cake

### 6.3.2. Sub-Visible Particulates

TABLE 1-48
Sub-visible particulates

| Sample | 10 μm (counts/vial) | 25 μm (counts/vial) | Pass/Fail |
|---|---|---|---|
| Formulation 1¹ | 855 | 71 | Pass |
| Formulation 2¹ | 3127 | 473 | Pass |
| Formulation 3¹ | 1866 | 46 | Pass |
| Formulation 4¹ | 5752 | 1304 | Fail |
| Formulation 5² | 3074 | 21 | Pass |

¹Where fill volume is 5.3 mL
²Where fill volume is 10.6 mL
Please note vials were not autoclaved/depyrogenated, this may cause the inflated readings in particulate matter.

The results for 10 μm are inflated for all formulations and formulations 2 and 4 are due to the adsorption occurring upon reconstitution. This coupled with lyophilization optimisation vials not undergoing the same cleaning procedure as tech batch vials have produced inflated results. All results do pass the pharmacopoeial specifications (<6000 particles per container, 10 μm and <600 particles per container) with the exception of formulation 4 that fails at the 25 μm level. This will be due to the poor reconstitution performance and adsorptive nature observed.

### 6.3.3. Water Content

TABLE 1-49
Water content

| | Mean water |
|---|---|
| Sample | (% w/w) |
| Formulation 1 | 2.60 |
| Formulation 2 | 2.87 |
| Formulation 3 | 11.08 |
| Formulation 4 | 1.69 |
| Formulation 5 | 3.84 |

All formulation produced optimal water levels of <5% with the exception of formulation 3 which was inflated. Formulation 3 required vigorous mixing and sonication due to poor dissolution in the dry methanol. The poor dissolution and increased mixing and sonication required for this batch suggests that this formulation does not perform well for moisture content and is highlighted in the inflated out of trend result in comparison to the other formulations.

### 6.3.4. Assay and Purity Results

### 6.3.4.1. Pre- and Post-Filtration

Samples were filtered using a 0.22 μm PES membrane syringe filter. No issues were observed during the filtration process before analysis via VWD detection.

TABLE 1-50
Pre and Post Filtration

| | Pre-Filtration | Post-Filtration | |
|---|---|---|---|
| | [BT5528] | [BT5528] | |
| Sample | (mg/mL) | (mg/mL) | % Recovery¹ |
| Formulation 1 | 3.92 | 3.94 | 100.5 |
| Formulation 2 | 3.71 | 3.76 | 101.5 |
| Formulation 3 | 3.70 | 3.54 | 95.8 |
| Formulation 4 | 3.66 | 3.82 | 104.4 |
| Formulation 5 | 1.96 | 2.02 | 103.5 |

¹% Recovery vs pre-filtration result.

TABLE 1-51
Purity/related substances Pre-Filtration

| | Formulation and amount (area %) | | | | |
|---|---|---|---|---|---|
| RRT | 1 | 2 | 3 | 4 | 5 |
| BT5528 | 98.41 | 99.01 | 99.25 | 99.25 | 99.20 |
| 1.10 | 1.51 | 0.98 | 0.26 | 0.75 | 0.72 |
| 1.11 | N.D | N.D | 0.49 | N.D | N.D |
| Total¹ | 1.5 | 1.0 | 0.8 | 0.8 | 0.7 |

¹Sum of related substances ≥ 0.10%

TABLE 1-52
Purity/related substances Post-Filtration

| | Formulation and amount (area %) | | | | |
|---|---|---|---|---|---|
| RRT | 1 | 2 | 3 | 4 | 5 |
| BR5528 | 99.24 | 99.38 | 99.11 | 99.13 | 99.30 |
| 1.10 | 0.76 | 0.52 | 0.89 | 0.87 | 0.63 |
| Total¹ | 0.8 | 0.5 | 0.9 | 0.9 | 0.6 |

¹Sum of related substances ≥ 0.10%

Recoveries were lower and out of trend for formulation 3 re-highlighting from the moisture and reconstitution details that this formulation is performing poorly in comparison to the other formulations. Trehalose is present in this formulation only which illustrates that this sugar is the root cause of the low recoveries and out of trend data.

### 6.3.4.2. Vial Content

TABLE 1-53
Lyophilized vial content

| | Vial 1 | Vial 2 | Mean | |
|---|---|---|---|---|
| | [BT5528] | [BT5528] | [BT5528] | |
| Sample | (mg/vial) | (mg/vial) | (mg/vial) | % Recovery¹ |
| Formulation 1 | 21.30 | 21.38 | 21.34 | 100.7 |
| Formulation 2 | 20.16 | 20.19 | 20.17 | 95.1 |
| Formulation 3 | 19.71 | 19.68 | 19.70 | 92.9 |
| Formulation 4 | 20.31 | 20.34 | 20.33 | 95.9 |
| Formulation 5 | 21.07 | 21.39 | 21.23 | 100.1 |

¹% Recovery vs theoretical 21.2 mg/vial BT5528.

Mean related substances are reported in Table 1-54.

TABLE 1-54
Purity/related substances

| | Formulation and amount (area %) | | | | |
|---|---|---|---|---|---|
| RRT | 1 | 2 | 3 | 4 | 5 |
| BT5528 | 99.30 | 99.13 | 99.24 | 99.30 | 99.22 |
| 1.10 | 0.70 | 0.87 | 0.76 | 0.70 | 0.79 |
| Total¹ | 0.7 | 0.9 | 0.8 | 0.7 | 0.8 |

¹Sum of related substances ≥ 0.10%

### 6.3.4.3. Purity/Related Substances

Formulation 1 was reconstituted with 3 separate volumes of WFI (5.3 mL, 10.6 mL and 15.9 mL) to give concentrations of 4 mg/mL, 2 mg/mL and 1.3 mg/mL respectively. Formulations 2, 3 and 4 were reconstituted in 5.3 mL WFI to give a concentration of 4 mg/mL and formulation 5 was reconstituted in 10.6 mL WFI to give a concentration of 2 mg/mL. The assay and purity were assessed and compared to the theoretical value.

TABLE 1-55
Reconstituted vial content

| | Assay | Rec./theory¹ |
|---|---|---|
| Sample | (mg/mL) | (%) |
| Formulation 1 (5.3 mL recon) | 3.52 | 88.1 |
| Formulation 1 (10.4 mL recon) | 1.02 | 50.9 |
| Formulation 1 (15.9 mL recon) | 1.32 | 99.2 |
| Formulation 2 | 3.55 | 88.8 |
| Formulation 3 | 2.51 | 62.8 |
| Formulation 4 | 3.42 | 85.5 |
| Formulation 5 | 2.06 | 103.0 |

¹% Recovery vs theoretical 4 mg/mL, 2 mg/mL and 1.33 mg/mL BT5528.

TABLE 1-56
Purity/related substances for Formulation 1 - three recon volumes

| | Reconstitution volume and amount (area %) | | |
|---|---|---|---|
| RRT | 5.3 mL | 10.6 mL | 15.9 mL |
| 0.98 | 0.12 | <LOQ | <LOQ |
| BT5528 | 99.08 | 98.69 | 98.95 |
| 1.10 | 0.32 | 0.54 | 0.25 |
| 1.11 | 0.44 | 0.21 | 0.26 |
| 1.12 | <LOQ | 0.53 | 0.58 |
| Total¹ | 0.9 | 1.3 | 1.1 |

TABLE 1-57
Purity/related substances Formulations 2-5

Formulation and amount (area %)

| RRT | 2 | 3 | 4 | 5 |
|---|---|---|---|---|
| 0.98 | N.D | <LOQ | <LOQ | 0.24 |
| BT5528 | 98.74 | 98.80 | 98.38 | 98.69 |
| 1.10 | 0.46 | 0.50 | 0.52 | 0.26 |
| 1.11 | 0.58 | 0.59 | 0.66 | 0.33 |
| 1.12 | 0.41 | <LOQ | 0.41 | 0.53 |
| Total¹ | 1.5 | 1.1 | 1.6 | 1.4 |

The assay values gained for formulations 1 to 5 were variable which can be expected from the initial reconstitution data, concluding that the BT5528 had adsorptive properties to the glass vial. Formulation 1 (1.3 mg/mL) and Formulation 5 produced the most optimal % recoveries (99.2% and 103.0%) respectively. The other % recoveries are attributable to the loss of drug product (API) from the reconstitution process. Particles adsorbed onto the glass vial and could not be fully dissolved further mixing and a stand period of approximately 1 hour. A stand period of 1 hour is an unrealistic procedure and for cycle optimisation 4 the stand period will be reduced to a smaller timeframe (˜5 minutes) to aid reconstitution.

### 6.4. Discussion and Conclusions

All formulations were shown to have an adsorptive nature and therefore variability and loss of product were seen throughout analytical testing for optimisation cycle 3. Formulation 5 performed better than the remaining four formulations assessed. Formulation 5 had a lower concentration of API (2 mg/mL) compared to the other formulations (4 mg/mL). It was recommended a lower concentration of API was assessed in optimisation cycle 4. This would enable the reconstitution performance to be further assessed and to achieve a consistent process for taking forward to the technical batch. It was also important the percentage assay recovery was consistently at target before the technical batch was manufactured.

### 7. Lyophilization Cycle 4

Due to the adsorptive nature of the formulation, additional formulations were assessed during lyophilization. From the data obtained during the sugar formulation screen, formulations 1 and 5 were reassessed with an additional formulation, Table 1-58. A lower (0° C.) secondary drying temperature was assessed during the sugar formulation screen to investigate potential over drying of the peptide and loss of the hydration shell. Lyophilization optimisation cycle 4 reverted back to the secondary drying temperature used previously of 25° C. as 0° C. did not improve the reconstitution characteristics.

The data from the sugar formulation screen produced an improved reconstitution profile at the lower BT5528 concentration of 2 mg/mL. Due to the reduction in adsorption for this presentation, Formulation 5 remained for optimisation cycle 4 with formulation 6 introduced with a lower concentration of polysorbate 20 to keep the ratio of polysorbate to the BT5528 API consistent with the original lead formulation (formulation 1). The compositions of the formulations are shown in Table 1-58.

TABLE 1-58
Drug product composition (Batch number and Formulation details)

| | |
|---|---|
| Formulation 1 | 4 mg/mL BT5528, 5.25 mg/mL histidine, 60 mg/mL, sucrose, 0.2 mg/mL polysorbate 20 |
| | (5.3 mL fill - 21.2 mg/vial) 20 mL vial |
| Formulation 5 | 2 mg/mL BT5528, 5.25 mg/mL histidine, 60 mg/mL, sucrose, 0.2 mg/mL polysorbate 20 |
| | (10.6 mL fill - 21.2 mg/vial) 20 mL vial |
| Formulation 6 | 2 mg/mL BT5528, 5.24 mg/mL histidine, 60 mg/mL, sucrose, 0.1 mg/mL polysorbate 20 |
| | (10.6 mL fill - 21.2 mg/vial) 20 mL vial |

### 7.1. Solution Preparation

Each of the formulations was prepared in the same manner. Approximately 35 mL or 70 mL of WFI was added to a beaker containing a magnetic stirrer bar. To this, 261.9 mg of histidine was added to Formulation 1 (50 mL), 524.9 mg to Formulation 5 and 524.7 mg to Formulation 6 (100 mL) with rinsing and stirred magnetically until dissolved. Once dissolved, 3.0011 g of sucrose was added to Formulations 1, 6.0010 g to Formulation 5 and 6.0064 g to Formulation 6 then stirred until dissolved. 1 mL of a 1% (w/v) Polysorbate 20 solution was pipetted into the histidine/sucrose solution for Formulation 1 and Formulation 6 and 2 mL to Formulation 5. The solutions were stirred until complete dissolution was achieved. The pH of each solution was measured.

To each of the three formulations, ˜257 mg of BT5528 was weighed out and added slowly then stirred for 90 minutes until fully dissolved. It should be noted that during addition/dissolution the API adhered to the side of the beaker for Formulation 1, this was less notable for Formulations 5 and 6 (4 mg/mL compared to 2 mg/mL). The API was removed from the side of the beaker using a pipette resulting in the API dissolving fully in the solution. The pH of the solutions was measure and adjusted to pH 6.5 with 1 M sodium hydroxide then transferred to a 50 mL (Formulation 1) or 100 mL (Formulations 5 and 6) volumetric flask and made to volume with WFI. The solutions were returned to the original beakers and stirred to mix.

Each solution was filtered through a single 0.22 μm PES syringe filter, the filtrate was a clear, colourless solution free from visible particulates.

### 7.2. Lyophilization

The solutions prepared were filled into 20 mL Type I clear glass vials at a volume of either 5.3 mL (Formulation 1) or 10.6 mL (Formulation 5 and Formulation 6), partially stoppered with 20 mm freeze drying stoppers and lyophilized directly from the shelf using the cycle in Table 1-59.

A single vial of each formulation located in the center of the tray was probed to monitor product temperature throughout the cycle. The cycle was monitored on the basis of temperature (product probe/shelf) and pressure (Capacitance manometer/Pirani gauge) differentials to determine the end point of primary and secondary drying.

TABLE 1-59
Lyophilization optimisation cycle 4

| | | Temperature | Pressure | Time | Ramp rate |
|---|---|---|---|---|---|
| Step | Cycle Stage | (° C.) | (mTorr) | (mins) | (° C./min) |
| 1 | Load | 5 | N/A | N/A | N/A |
| 2 | Hold | 5 | N/A | 30 | N/A |
| 3 | Ramp | −40 | N/A | 180 | 0.25 |
| 4 | Freezing | −40 | N/A | 180 | N/A |
| 5 | Extra freezing | −40 | 100 | 30 | N/A |
| 6 | Ramp | −25 | 100 | 75 | 0.20 |
| 7 | Primary drying | −25 | 100 | 6386 | N/A |
| 8 | Ramp | 25 | 20 | 250 | 0.20 |
| 9 | Secondary drying | 25 | 20 | 1102 | N/A |

| | | |
|---|---|---|
| 10 | Finish | Vials stoppered to 722,000 mTorr with (pure) nitrogen |

Total cycle duration ~137 hrs (~5.7 days)

### 7.3. Results

### 7.3.1. Appearance of Solution, pH and Reconstitution Time

TABLE 1-60
Appearance of solution, pH reconstitution time

| | | | Reconstitution time |
|---|---|---|---|
| Sample | Appearance¹ | pH | (minutes) |
| Formulation 1 | A clear colourless solution, moderate adsorption noted on side walls. | 6.6 | 1 mins 52 secs |
| Formulation 5 | A clear colourless solution, less adsorption on side walls³ | 6.6 | 39 secs |
| Formulation 6 | A clear colourless solution, less adsorption on side walls² | 6.6 | 40 sees |

¹All formulaitons had visible particles. See FIG. 32 for photographs of reconstituted solution.

As before, it was more difficult to fully reconstitute Formulation 1 which has 4 mg/mL BT5528. Formulations 5 and 6 were easier to reconstitute due to the lower BT5528 concentration. Formulation 1 produced a higher degree of adsorption than formulations 5 and 6 highlighting that the reduced 2 mg/mL BT5528 concentration produces a better reconstitution and subsequently less adsorption occurs with only very small particles adsorbing.

TABLE 1-61
Appearance lyophilized plug

| Sample | Appearance |
|---|---|
| Formulation 1 | A homogenous white lyophilised cake |
| | with evident shrinkage |
| Formulation 5 | A homogenous white lyophilised cake |
| | with evident shrinkage |
| Formulation 6 | A homogenous white lyophilised cake |
| | with evident shrinkage |

### 7.3.2. Sub-Visible Particulates

TABLE 1-62
Sub-visible particulates

| | 10 μm | 25 μm | |
|---|---|---|---|
| Sample | (counts/vial) | (counts/vial) | Pass/Fail³ |
| Formulation¹ | 191 | 18 | Pass |
| Formulation 5² | 445 | 92 | Pass |
| Formulation 6² | 247 | 35 | Pass |

¹Where fill volume is 5.3 mL
²Where fill volume is 10.6 mL
³Number particles with diameter ≥10 μm per vial ≤6000 = PASS and number particles with diameter ≥25 μm per vial ≤600 = PASS

The results for 10 μm are slightly inflated for all formulations. The lyophilization optimisation vials do not undergo the same cleaning procedure as tech batch vials and may produce inflated results.

All results pass the pharmacopoeia specifications—number of particles with diameter ≥10 μm per vial ≤6000 and number particles with diameter ≥25 μm per vial ≤600.

7.3.3. Water Content

The moisture content of the lyophilized plugs was determined by Karl Fisher analysis using methanol as the solvent. Single samples from duplicate vials were analyzed per batch, Table 1-63. No reconstitution issues were noted during the addition of dry methanol.

TABLE 1-63
Water content

| | Mean water |
|---|---|
| Sample | (% w/w) |
| Formulation 1 | 0.71 |
| Formulation 5 | 1.51 |
| Formulation 6 | 1.47 |

All formulation produced optimal water levels of <5%. No reconstitution issues noted during the addition of dry methanol.

### 7.3.4. Assay and Purity Results

### 7.3.4.1. Pre-Filtration

Samples were filtered using a 0.22 μm PES membrane syringe filter. No issues were observed during the filtration process. In error post-filtration samples were not collected prior to lyophilization hence results are reported against the theoretical concentration.

TABLE 1-64
Pre and Post Filtration

| | Pre-Filtration | |
|---|---|---|
| | [BT5528] | |
| Sample | (mg/mL) | % Recovery¹ |
| Formulation 1 | 3.64 | 91.0 |
| Formulation 5 | 1.97 | 98.5 |
| Formulation 6 | 1.91 | 95.5 |

¹% Recovery vs theoretical valued 4 mg/mL for formulation 1 and 2 mg/mL for formulation 5 and 6.

TABLE 1-65
Purity/related substances Pre-Filtration

| | Formulation and amount (area %) | | |
|---|---|---|---|
| RRT | 1 | 5 | 6 |
| 0.51 | 0.22 | 0.18 | 0.22 |
| 0.53 | 0.22 | 0.21 | 0.27 |
| 0.97 | 0.13 | 0.13 | 0.11 |
| BT5528 | 98.35 | 98.63 | 98.61 |
| 1.10 | 0.55 | 0.61 | 0.51 |
| 1.30 | 0.11 | 0.11 | 0.14 |
| Total¹ | 1.5 | 1.2 | 1.3 |

¹Sum of related substances ≥0.10%

Formulations 5 and 6 have greater than 95% recovery versus the theoretical 2 mg/mL concentration. The recovery value for Formulation 1 is considerably lower at 91%, suggesting that a 2 mg/mL presentation is likely to be more robust.

The related substances data is consistent for all three formulations.

### 7.3.5. Vial Content

TABLE 1-66
Lyophilized vial content

| | Vial 1 | Vial 2 | Mean | |
|---|---|---|---|---|
| | [BT5528] | [BT5528] | [BT5528] | |
| Sample | (mg/vial) | (mg/vial) | (mg/vial) | % Recovery¹ |
| Formulation 1 | 19.75 | 19.65 | 19.70 | 92.9 |
| Formulation 5 | 21.53 | 21.74 | 21.63 | 102.0 |
| Formulation 6 | 21.11 | 20.92 | 21.01 | 99.1 |

¹% Recovery vs theoretical 21.2 mg/vial BT5528.

Mean related substances are reported in Table 1-67.

TABLE 1-67
Purity/related substances

| | Formulation and amount (area %) | | |
|---|---|---|---|
| RRT | 1 | 5 | 6 |
| 0.51 | 0.19 | 0.18 | 0.17 |
| 0.53 | 0.26 | 0.24 | 0.23 |
| 0.97 | 0.15 | 0.15 | 0.14 |
| BT5528 | 98.34 | 98.26 | 98.44 |
| 1.10 | 0.70 | 0.79 | 0.65 |
| 1.30 | 0.10 | 0.11 | 0.12 |
| Total¹ | 1.4 | 1.5 | 1.3 |

¹Sum of related substances ≥0.10%

### 7.3.6. Reconstituted Solution

Formulation 1 was reconstituted with 5.3 mL WFI to give a concentration of 4 mg/mL. Formulations 5 and 6 were reconstituted in 10.6 mL WFI to give a concentration of 2 mg/mL. The assay and purity were assessed and compared to the theoretical value.

TABLE 1-68
Reconstituted vial content

| | Vial 1 | Vial 2 | Mean | |
|---|---|---|---|---|
| | [BT5528] | [BT5528] | [BT5528] | |
| Sample | (mg/mL) | (mg/mL) | (mg/mL) | % Recovery¹ |
| Formulation 1 | 2.98 | 3.25 | 3.11 | 77.8 |
| Formulation 5 | 1.84 | 1.88 | 1.86 | 93.0 |
| Formulation 6 | 1.79 | 1.84 | 1.81 | 90.5 |

¹% Recovery vs theoretical 4 mg/mL and 2 mg/mL BT5528.

Mean related substances results are reported in Table 1-69.

TABLE 1-69
Purity/related substances Formulations 1, 5 and 6.

| | Formulation and amount (area %) | | |
|---|---|---|---|
| RRT | 1 | 5 | 6 |
| 0.51 | 0.28 | 0.23 | 0.20 |
| 0.53 | 0.31 | 0.29 | 0.27 |
| 0.97 | 0.15 | 0.14 | 0.12 |
| BT5528 | 98.42 | 98.58 | 98.53 |
| 1.10 | 0.58 | 0.53 | 0.48 |
| 1.30 | 0.12 | 0.11 | 0.15 |
| Total¹ | 1.4 | 1.3 | 1.2 |

Related substances data is consistent for all three formulations.

The assay data in Table 1-69 is lower than the target for all three formulations. It is notably worse for formulation 1 which has a recovery of approximately 78%. Formulation 1 has the highest BT5528 concentration at 4 mg/mL and hence was the most difficult to reconstitute fully due to the nature of the BT5528 API. The results indicate that the lower 2 mg/mL BT5528 concentration is a more favorable presentation and should be considered for the non-GMP technical batch.

### 7.4. Conclusions

The appearance of the lyophilized was similar for all three formulations assessed, white, homogeneous with slight shrinkage. The change in BT5528 concentration and higher fill volume did not affect the appearance of the product.

The reconstitution time of the 2 mg/mL solutions (Formulation 5 and 6) were over 1 minute faster than the 4 mg/mL solution (Formulation 1). There was no difference in the reconstitution time of the two Polysorbate 20 concentrations, 0.1 and 0.2 mg/mL (Formulations 5 and 6 respectively), indicating the Polysorbate 20 did not affect the reconstitution time.

The moisture content of Formulation 1 was lower than Formulations 5 and 6 which would be expected as the fill volume was half.

There was no difference in the purity/related substances pre- and post-lyophilization or between the three formulations assessed. There was, however, a difference in percentage recovery vs theoretical. The assay value for Formulation 1 was 77.8% of theoretical (3.11 mg/mL) compared to 91-93% for Formulations 5 and 6 (1.86 and 1.18 mg/mL) showing the improved reconstitution of lyophilized product with a lower BT5528 concentration.

### 8. Filtration Assessment

During GMP manufacture the bulk solution would be filter sterilized (0.22 μm pore size). Compatibility with the filter and possible loss of material by adsorption onto the filter membrane and housing should be investigated. Related substances would also be monitored.

A known volume of the formulation (Table 1-71) was passed, by peristaltic pump and platinum-cured silicone tubing, through proprietary “P” (pharmaceutical) grade filter capsule of known surface area to assess adsorptive losses per cm2 of filter surface area on a single type of membrane; PES (polyether sulfone) (Mini Kleenpak capsule filter, KA02EKVP2S, surface area of 220 cm²).

Ease of filtration, measured as the back pressure up-stream of the filter, was assessed. Active concentration and related substances in the pre-filter solution and in five successive early samples of the filtrate and in the final bulk filtrate were determined by UPLC. The pH of the pre-filter and filtrate samples was also determined.

Adsorption of active material onto the filter membrane or capsule surface, if it occurs, is usually a saturable phenomenon. Having identified the volume of solution to give saturation of a particular type of filter capsule, this becomes the initial discard volume for that particular capsule. A useful in-house arrangement is to ensure that the ratio of filtered volume (mL) to total filter surface (cm²) is ≥5.

TABLE 1-70
Filtration assessment formulation composition (Batch number and Formulation details)

| | |
|---|---|
| Filtration Assessment | 2 mg/mL BT5528, 5.24 mg/mL histidine HCl, 60 mg/mL sucrose, 0.1 mg/mL polysorbate |
| | 20 (10.6 mL fill - 21.2 mg/vial) 20 mL vial |

### 8.1. Solution Preparation

The formulation detailed in Table 1-70 was prepared on a 200 mL scale. Approximately 140 mL of WFI was added to a beaker containing a magnetic stirrer bar. To this, ˜1.05 g of histidine was added with rinsing and stirred magnetically until dissolved. Once dissolved, 12 g of sucrose was weighed out and added with rinsing then stirred until dissolved. 2 mL of a 1% (w/v) Polysorbate 20 solution was pipetted into the histidine/sucrose solution and stirred until complete dissolution was achieved.

To the solution, ˜514 mg of BT5528 was weighed out and added slowly then stirred for 90 minutes until fully dissolved. The pH of the solution was measured and adjusted to pH 6.5 with 1 M sodium hydroxide then transferred to a 200 mL volumetric flask and made to volume with WFI. The solution was returned to the original beaker and stirred to mix.

8.2. Filtration

A sample of the solution was retained pre-filtration for UPLC analysis, and the remainder filtered through a single 0.22 μm PES capsule filter using platinum-cured silicone tubing and a peristaltic pump.

Aliquots were collected throughout the filtration process as follows:
Pre-filtration
0-10 mL
10-20 mL
20-30 mL
30-40 mL
40-50 mL
Bulk filtrate

The solution filtered with ease and the back pressure was low, the resultant filtrate was a clear, colourless solution free from visible particulates.

The aliquots were analyzed by UPLC for assay and related substances to determine if there was a loss of assay on the filter membrane or extractables/leachables indicating incompatibility with the membrane.

### 8.3. Results

TABLE 1-71
Filtration samples vial content

| | Assay | Rec./theory¹ | Rec./pre-filt² |
|---|---|---|---|
| Sample | (mg/mL) | (%) | (%) |
| Pre-Filtration | 2.03 | 101.5 | — |
| First few mL | 1.77 | 88.5 | 87.2 |
| 10-20 mL | 1.97 | 98.5 | 97.0 |
| 20-30 mL | 1.98 | 99.0 | 97.5 |
| 30-40 mL | 2.02 | 101.0 | 99.5 |
| 40-50 mL | 2.06 | 103.0 | 101.5 |
| Bulk Solution | 2.04 | 102.0 | 100.5 |

¹% Recovery vs theoretical 2 mg/mL BT5528.
²% Recovery vs. pre-filtration result

TABLE 1-72
Purity/related substances filtration assessment

| | Volume and amount (area %) | | | | | | |
|---|---|---|---|---|---|---|---|
| | | First | 10-20 | 20-30 | 30-40 | 40-50 | |
| RRT | Pre | few mL | mL | mL | mL | mL | Bulk |
| 0.51 | 0.18 | 0.21 | 0.19 | 0.18 | 0.18 | 0.19 | 0.18 |
| 0.53 | 0.22 | 0.26 | 0.21 | 0.23 | 0.20 | 0.21 | 0.21 |
| 0.96 | 0.14 | 0.11 | 0.12 | 0.13 | 0.13 | 0.15 | 0.14 |
| 0.97 | 0.56 | 0.65 | 0.60 | 0.59 | 0.61 | 0.59 | 0.55 |
| BT5528 | 97.94 | 98.18 | 98.28 | 98.17 | 98.16 | 98.11 | 98.27 |
| 1.10 | 0.61 | 0.47 | 0.57 | 0.64 | 0.65 | 0.67 | 0.46 |
| Total¹ | 1.8 | 1.7 | 1.7 | 1.8 | 1.8 | 1.8 | 1.5 |

A drop in assay was noted during the first 0-10 mL of sample passed through the PES filter. This is not unexpected and indicated that a small discard volume is necessary for this filter type. Acceptable recoveries were achieved after 0-10 mL had been filtered and there was no significant change in total impurities.

PES filters are therefore appropriate for the preparation of BT5528 drug product.

### 8.4. Discussion and Conclusions

The solution filtered through the PES capsule filter with a low back pressure indicating the membrane was suitable for filtration of this solution.

There was no change in the purity/related substances data in the aliquots collected or in the bulk solution indicating no extractable/leachables were present.

The assay data showed an initial decrease in the BT5528 concentration in the first aliquot collected (0-10 mL) of 88.5% recovery vs theoretical, however, the assay returned to target after 10-20 mL had been filtered. This indicated a discard volume of 10 mL per filter should be employed during the non-GMP technical batch.

### 9. Conclusions

This report has demonstrated the successful development of a stable lyophilized formulation for progression to a non-GMP technical batch.

Lyophilization cycle optimisation was carried out, however, due to issues with BT5528 adsorption to the surface of the vials and challenges with reconstitution a more conservative cycle was progressed to the non-GMP technical batch.

A filtration assessment was undertaken which showed an initial decrease in the BT5528 concentration during filtration which returned to target after 10 mL. A discard volume of 10 mL per filter will be employed during the non-GMP technical batch.

The cause of adsorption of BT5528 to the surface of the vials was investigated during separate lyophilization cycles. A number of hypotheses were presented as potential causes, high sodium chloride concentration in the final product, a more basic pH of the reconstituted product, silanised vials and over-drying of the peptide. Each of these hypotheses were assessed together with a formulation screen of alternative sugars, surfactant and a lower BT5528 concentration to investigate if this improved the reconstitution characteristics. An improvement in the reconstitution time was obtained with a lower pH pre-lyophilization, however, oily droplets remained on the surface of the vial.

A reduction in the sodium chloride content, use of silanised vials and a lower secondary drying temperature (to increase the final moisture content of the product) did not improve the reconstitution characteristics. Alternative sugars or surfactant did not generate an improvement, however, a reduction in BT5528 concentration from 4 mg/mL to 2 mg/mL was found to improve the reconstitution characteristics. The reconstitution time was reduced and no oily droplets were observed on the surface of the vials post-reconstitution. The recovery vs theoretical was at target in the 2 mg/mL formulation compared to consistently below target for the 4 mg/mL formulations.

Based on the results of the investigations into BT5528 surface adsorption, and after discussions with the client, a lead formulation with a BT5528 concentration of 2 mg/mL was selected for progression to the non-GMP technical batch.

### Example 2. Phase I/II Study of the Safety, Pharmacokinetics, and Preliminary Clinical Activity of BT5528 in Patients with Advanced Malignancies Associated with EphA2 Expression

### 2.1 Objectives

### Primary Objectives

The primary objectives of the escalation (Parts A-1 and A-2) are:
To assess safety and tolerability of BT5528 in patients with advanced solid tumor malignancies associated with EphA2-expression as a monotherapy (Part A-1) and in combination with nivolumab (Part A-2)
To define the maximum tolerated dose (MTD) of BT5528, if observed, and determine a recommended Phase II dose (RP2D) as a monotherapy (Part A-1) and in combination with nivolumab (Part A-2).

The primary objectives of the expansions (Parts B-1 and B-2) are:
To assess the clinical activity of BT5528 in patients with selected solid tumor indications as a monotherapy (Part B-1) and in combination with nivolumab (Part B-2) using RECIST 1.1

### Secondary Objectives

The secondary objectives of the escalation (Parts A-1 and A-2) this study are:
To assess preliminary signals of anti-tumor activity achieved with BT5528 administration in patients with advanced solid tumor malignancies associated with EphA2-expression as a monotherapy (Parts A-1) and in combination with nivolumab (Parts A-2)
To determine pharmacokinetic (PK) parameters of BT5528
To determine incidence of anti-drug antibody (ADA) development

The secondary objectives of the expansions (Parts B-1 and B-2) study are:
To assess safety and tolerability of BT5528 in patients with selected solid tumor indications as a monotherapy (Part B-1) and in combination with nivolumab (Part B-2)
To determine pharmacokinetic (PK) parameters of BT5528
To determine incidence of anti-drug antibody (ADA) development

### 2.2. Study Design

This study is a Phase I/II, first-in-human, open-label dose-escalation study of BT5528 given as a single agent (Parts A-1 and B-1) and in combination with nivolumab (Parts A-2 and B-2). There are two parts to this study: Part A, dose escalation and Part B, dose expansion.

### 2.3. Study Drugs, Doses, and Modes of Administration:

BT5528, in ascending doses, administered intravenously as infusion over 1 h. Nivolumab administered as per label.

### 2.4. Inclusion Criteria—All Patients:

Patients must meet the following criteria in order to be included in the research study:
1. Written informed consent, according to local guidelines, signed and dated by the patient or by a legal guardian prior to the performance of any study-specific procedures, sampling, or analyses.
   If a patient declines to participate in any voluntary component of the study (e.g., tumor biopsy), there will be no penalty or loss of benefit to the patient and he/she will not be excluded from other aspects of the study.
2. At least 18 years-of-age at the time of signature of the informed consent form
3. Eastern Cooperative Oncology Group (ECOG) Performance Status score of 0 or 1

| ECOG Performance Status Scale | | Karnofsky Performance Scale | |
|---|---|---|---|
| Grade | Descriptions | Percent | Description |
| 0 | Normal activity. Fully active, able to | 100 | Normal, no complaints, no evidence of |
| | carry on all pre-disease performance | | disease. |
| | without restriction. | 90 | Able to carry on normal activity: minor |
| | | | signs or symptoms of disease. |
| 1 | Symptoms, but ambulatory. Restricted | 80 | Normal activity with effort; some signs |
| | in physically strenuous activity, but | | or symptoms of disease. |
| | ambulatory and able to carry out work | 70 | Cares for self, unable to carry on |
| | of a light or sedentary nature (e.g., light | | normal activity or to do active work. |
| | housework, office work). | | |

4. Patients must have measurable disease per Response Evaluation Criteria in Solid Tumors (RECIST) v1.1,
5. Acceptable organ function, as evidenced by the following laboratory data:
   Renal function, as follows: creatinine clearance of ≥50 mL/min by the Cockcroft-Gault equation or as measured by 24-hour urine collection.
   Total bilirubin ≤1.5×ULN (upper limit of normal)
   Serum albumin ≥2.5 g/dL
   Aspartate aminotransferase (AST) ≤2.5×ULN or ≤5×ULN in the presence of liver metastases
   Alanine aminotransferase (ALT) ≤2.5×ULN or ≤5×ULN in the presence of liver metastases
   International normal ratio (INR) <1.3 or ≤institutional ULN (anticoagulants not allowed)
6. Acceptable hematologic function (no red blood cell or platelet transfusions or growth factors are allowed within 4 weeks of the first dose of BT5528):
   Hemoglobin ≥9 g/dL
   Absolute neutrophil count (ANC) ≥1500 cells/mm³
   Platelet count ≥75,000 cells/mm³
7. Negative pregnancy test for women of childbearing potential (WOCBP) (negative serum test at screening and negative urine or serum test within 3 days prior to the first dose of BT5528. Male patients with female partners of childbearing potential and female patients of childbearing potential are required to follow highly effective contraception (oral and hormonal contraceptives allowed) at least as conservative as Clinical Trial Facilitation Group (CTFG) recommendations for less than 1% failure rate (https://www.hma.eu/fileadmin/dateien/Human_Medicines/01-About_HNA/Working_Groups/CTFG/2014_09_HMA_CTFG_Contraception.pdf), during their participation in the study and for 6 months following last dose of study drug. Male patients must also refrain from donating sperm during their participation in the study for 6 months following last dose of study drug and women must not breastfeed during that time or donate eggs.
   Women Not of Childbearing Potential are defined as Follows:
      Women are considered post-menopausal and not of childbearing potential if they have had 12 months of natural (spontaneous) amenorrhea with an appropriate clinical profile (e.g., age appropriate, history of vasomotor symptoms).
      Women who are permanently sterilized (e.g., tubal occlusion, hysterectomy, bilateral salpingectomy, bilateral oophorectomy).
      Women who are >45 years-of-age, not using hormone-replacement therapy and who have experienced total cessation of menses for at least 12 months OR who have a follicle stimulating hormone (FSH) value >40 mIU/mL and an estradiol value <40 pg/mL (140 pmol/L).
      Women who are >45 years-of-age, using hormone-replacement therapy and who have experienced total cessation of menses for at least 1 year OR who have had documented evidence of menopause based on FSH>40 mIU/mL and estradiol <40 pg/mL prior to initiation of hormone-replacement therapy.
8. Availability of archived tumor samples within 9 months prior to the date of the first dose of BT5528 or willingness to provide fresh tumor biopsy during screening.
9. Life expectancy ≥12 weeks after the start of BT5528 treatment according to the Investigator's judgment.
10. Must be willing and able to comply with the protocol and study procedures.

### Additional Inclusion Criteria—Part A Only

1. Patients with advanced, histologically confirmed malignant solid tumors historically known for high expression of EphA2 (non-small-cell lung cancer (NSCLC), ovarian cancer, triple-negative breast cancer (TNBC), gastric/upper gastrointestinal (GI), pancreatic and urothelial cancers), that recurred after previous therapy and are candidates for a Phase I study due to lack of approved or standard options for treatment. Patients with other tumors may be enrolled if they provide evidence of high expression of EphA2 on tumor tissue collected within 9 months prior to the date of the first dose of BT5528. The SRC may decide to require enrollment of specific tumor types among those listed in inclusion 3.1.1 at any point during the escalation if it is felt necessary to enrich the evaluation of biomarkers, safety or PK in a specific tumor type.

### Additional Inclusion Criteria—Part B-1 and B-2 NSCLC Monotherapy and Combination Cohorts

1. Patients with metastatic recurrent disease histologically confirmed to be an adenocarcinoma subtype of NSCLC (adeno-NSCLC) are eligible and must have exhausted all standard treatment options including progression on or after platinum-based chemotherapy, must have failed at least one prior line of therapy with evidence of radiographic progression on the most recent line of therapy. If EGFR, ALK, NTRK, ROS1 or other genomic tumor aberrations, must have not been a candidate for or must have received appropriate treatment for driver mutation disease, if applicable. If prior immunotherapy, the last dose must have been at least 28 days prior to the first dose of BT5528.
2. At least 6 patients per cohort must have at least 1 tumor lesion amenable to biopsy and must be willing to undergo a biopsy prior to first dose of BT5528 and following any dose in Cycle 1.

### 2.5. Exclusion Criteria—All Patients:

Patients who meet any of the following criteria will be excluded from study entry:
1. Chemotherapy treatments within 14 days prior to first dose of study treatment, other anticancer treatments, treatment within 28 days or 5 half-lives, whichever shorter. Prior toxicities must have resolved to grade 1 per Common Terminology Criteria for Adverse Events (CTCAE) v 5.0 (except alopecia which must be no greater than Grade 2).
2. Experimental treatments within 4 weeks of first dose of BT5528.
3. Current treatment with strong inhibitors or inducers of CYP3A4 or strong inhibitors of P-gp including herbal- or food-based.
4. Known sensitivity to any of the ingredients of the investigational product or monomethyl auristatin E (MMAE).
5. Significant medical condition, life-threatening illness, active uncontrolled infection or organ system dysfunction (such as ascites, coagulopathy, encephalopathy), or other reasons which, in the Investigator's opinion, could compromise the patient's safety, or interfere with or compromise the integrity of the study outcomes including consideration of gastrointestinal, skin and pulmonary co-morbidities and including review of screening chest CT to ensure no clinically significant co-morbidities.
6. Major surgery (excluding placement of vascular access) within 4 weeks of first dose of BT5528 and must have recovered adequately prior to starting study therapy
7. Receipt of live vaccine within 30 days of study treatment
8. Uncontrolled, symptomatic brain metastases (must have stable neurologic status following local therapy for at least 4 weeks without the use of steroids or on stable or decreasing dose of less than or equal to 10 mg daily prednisone or equivalent and must be without neurologic dysfunction that would confound the evaluation of neurologic and other AEs.)
9. Patients with uncontrolled hypertension (systolic blood pressure [BP]≥139 mmHg; diastolic BP≥89 mmHg) prior to first dose of BT5528 (must have been in stable control for at least 3 months)
10. History or current evidence of any condition, therapy or laboratory abnormality that might confound the results of the study, interfere with the patient's participation, or is not in the best interest of the patient to participate in the opinion of the Investigator including but not limited to:
   Patients with history of a cerebral vascular event (stroke or transient ischemic attack), unstable angina, myocardial infarction, congestive heart failure or symptoms of New York Heart Association Class III-IV documented within 6 months prior to first dose of BT5528 or:
      i. Mean resting corrected QT interval (QTcF) >470 msec
      ii. Any factors that increase the risk of QTc prolongation or risk of arrhythmic events such as heart failure, hypokalemia, congenital long QT syndrome, family history of long QT syndrome or unexplained sudden death under 40 years-of-age, or any concomitant medication known to prolong the QT interval
      iii. Any clinically important abnormalities (as assessed by the Investigator) in rhythm, conduction, or morphology of resting electrocardiograms (ECGs), e.g., complete left bundle branch block, third degree heart block
11. Known human immunodeficiency virus (HIV) or acquired immune deficiency syndrome (AIDS)
12. Patients with a positive hepatitis B surface antigen and/or anti-hepatitis B core antibody. Patients with a negative polymerase chain reaction (PCR) assay are permitted with appropriate antiviral therapy
13. Active hepatitis C infection with positive viral load if hepatitis C virus (HCV) antibody positive (if antibody is negative then viral load not applicable). Patients who have been treated for hepatitis C infection can be included if they have documented sustained virologic response of ≥12 weeks.
14. Thromboembolic events and/or bleeding disorders within 3 months (e.g., deep vein thrombosis [DVT] or pulmonary embolism [PE]) prior to the first dose of BT5528.
15. History of another malignancy within 3 years before the first dose of BT5528, or any evidence of residual disease from a previously diagnosed malignancy (excluding adequately treated with curative intent basal cell carcinoma, squamous cell of the skin, cervical intraepithelial neoplasia/cervical carcinoma in situ or melanoma in situ or ductal carcinoma in situ of the breast).
16. Systemic anti-infective treatment or fever within the last 14 days prior to first dose of BT5528.
17. Psychological, familial, sociological, or geographical conditions that do not permit compliance with the protocol and/or follow-up procedures outlined in the protocol.

### Additional Exclusion Criteria Part A-2 and B-2 Nivolumab Combination Cohorts

1. Prior intolerance to immune checkpoint inhibitor
2. Known hypersensitivity to checkpoint inhibitor therapy
3. Prior organ transplant (including allogeneic)
4. Diagnosis of clinically relevant immunodeficiency
5. Active systemic infection requiring therapy
6. More than 10 mg daily prednisone equivalent or other strong immunosuppressant
7. History of autoimmune disease except alopecia or vitiligo
8. History of interstitial lung disease

### 2.6 Correlative Testing:

All patients will be required to provide archive tumor material or fresh tumor biopsy for assessment of expression levels of EphA2 and additional molecular genetic characterization (i.e. assessment of specific somatic mutations, etc.). This material should be provided as a tissue block or 10-15 paraffin-dipped unstained slides.

Pre- and post-dose tumor biopsies will be collected to investigate intratumoral PK/Pharmacodynamic effects of BT5528. Pre- and one post-dose tumor biopsy will be optional for all patients but will be mandatory for a subset of patients in Part B (6 per cohort). The post-dose biopsy will be required in Cycle 1 after any dose as long as it is within 4 to 36 hours after the BT5528 dose. Refer to the schedule of assessments (SOA) for further details.

Pre- and post-dose blood samples will also be collected to assess pharmacodynamic, response, and treatment resistance biomarkers, such as somatic mutations in circulating tumor DNA (ctDNA), ADA and pharmacogenomic analysis.

### 2.7. Statistical Methodology:

Dose-escalation (applies separately to A-1 and A-2): The actual number of dose levels to be explored in this study will depend on determination of the non-tolerable dose based on dose-limiting toxicities (DLTs). The MTD will be defined based on DLTs (see Section 5). Other safety data, as well as PK profiles observed during the conduct of the study and any trends for anti-tumor activity will be used to determine the RP2D which will be no greater than the MTD.

A 3+3 design will be used for the first two dose levels. At least 3 evaluable patients will be enrolled at each dose level and will be evaluated for 28 days before escalation to the next dose level can occur. After confirmation of tolerability in dose level 1, dose escalation of no more than 100% will be allowed to dose level 2. Treatment cycles will occur consecutively as per the SOA. If one patient experiences a DLT an additional 3 patients will be treated with the same dose. Evaluation of a cohort of at least 3 patients completing 1 cycle of treatment (28 days) is required prior to proceeding to the next dose level. Additional details are found in Section 5.

Following evidence of tolerability at the first two dose levels, all subsequent dose interval escalations will be based on a type of continual reassessment method (CRM) using a two-parameter Bayesian logistic regression model (BLRM) and the escalation with overdose control (EWOC) principle that the next higher escalated dose level will include the highest posterior probability of a DLT occurring in the target interval (20%, 33%) among doses fulfilling the overdose criterion that there exist ≤25% likelihood of the dose level being found unsafe (DLT rate ≥33%). The BLRM will be applied to cumulative DLT/safety data and results will be made available to the SRC and, upon review of these data, the SRC will make a recommendation regarding the precise dose escalation. An estimated escalation scheme is provided in section 5, with full details in Appendix F: Details and Operating Characteristics of the Dose Escalation Design.

Each cohort of Part B will employ a Simon 2-stage design where p0=0.175 and p1=0.35 with a 1-sided alpha of 0.05 and 80% power, where p0 and p1 are the null and alternative hypotheses for the overall response rate (ORR). If 3 or more patients have an objective response among the first 14 patients recruited (≥21% ORR) a further 26 patients will be dosed; otherwise, the cohort will be stopped.

Therefore, the maximum number of patients recruited into the study is 152; 48 from Part A-1, 24 from Part A-2 and 40 from each of the two Part B cohorts.

1. A pharmaceutical composition comprising BT5528, or a pharmaceutically acceptable salt thereof, and a pharmaceutically acceptable excipient or carrier selected from a buffering agent, a filler or extender or binder, and a surfactant.

2. The pharmaceutical composition of claim 1, wherein the buffering agent is histidine hydrochloride.

3. The pharmaceutical composition of claim 1 or 2, wherein the filler or extender or binder is sucrose.

4. The pharmaceutical composition of any one of claims 1-3, wherein the surfactant is polysorbate-20.

5. The pharmaceutical composition of any one of claims 1-4, further comprising an isotonicity adjusting agent.

6. The pharmaceutical composition of claim 5, wherein the isotonicity adjusting agent comprises dextrose.

7. The pharmaceutical composition of any one of claims 1-6, which is a solid pharmaceutical composition in lyophilized powder form.

8. The pharmaceutical composition of any one of claims 1-6, which is a liquid pharmaceutical composition further comprising water.

9. The pharmaceutical composition of any one of claims 1-8, comprising:
BT5528, or a pharmaceutically acceptable salt thereof;
about 1.31-2.62 mg histidine hydrochloride per mg of BT5528, or a pharmaceutically acceptable thereof;
about 15-30 mg sucrose per mg of BT5528, or a pharmaceutically acceptable thereof; and
about 0.05-0.1 mg Polysorbate 20 per mg of BT5528, or a pharmaceutically acceptable thereof.

10. The pharmaceutical composition of claim 9, comprising:
about 21.2 mg BT5528, or a pharmaceutically acceptable salt thereof;
about 55.5 mg histidine hydrochloride per mg of BT5528, or a pharmaceutically acceptable thereof;
about 636 mg sucrose per mg of BT5528, or a pharmaceutically acceptable thereof; and
about 1.06-2.12 mg Polysorbate 20 per mg of BT5528, or a pharmaceutically acceptable thereof.

11. The pharmaceutical composition of claim 8, comprising:
about 2-4 mg/mL BT5528, or a pharmaceutically acceptable salt thereof;
about 5.25 mg/mL histidine hydrochloride;
about 60 mg/mL sucrose; and
about 0.1-0.2 mg/mL Polysorbate 20.

12. A method for treating an advanced solid tumor malignancy associated with EphA2-expression in a patient comprising intravenously administering to the patient the pharmaceutical composition of any one of claims 1-11.

13. The method of claim 12, wherein the advanced solid tumor malignancy associated with EphA2-expression is selected from non-small-cell lung cancer (NSCLC), ovarian cancer, triple-negative breast cancer (TNBC), gastric/upper gastrointestinal (GI), pancreatic and urothelial cancers.

14. The method of claim 12 or 13, wherein the pharmaceutical composition is administered once every 7 days.

15. The method of any one of claims 12-14, wherein the pharmaceutical composition is administered at a dose of about 2.2, 4.4, 7.3, 11, 14.6, or 19.4 mg/m².

16. The method of any one of claims 12-15, wherein the pharmaceutical composition is administered via an IV infusion of about 60 minutes.

17. The method of any one of claims 12-15, further comprising administering Nivolumab.
